SEQUENCE LISTING (112 sequences)

SEQ ID NO: 1 - DNA, 77 bp, Artificial Sequence
  Synthetic
tgccgctagaagcaggcgtcacagtgcccccagagtgcttgtcagtcttagaaacagaggcatcggaagacaagcgt

SEQ ID NO: 2 - DNA, 84 bp, Artificial Sequence
  Synthetic
gctagtcacagcctggcgcctggtgtcccctcccttcccaagccccctcagcttttccactgccaccggcgtacaagcaagtgc

SEQ ID NO: 3 - DNA, 78 bp, Artificial Sequence
  Synthetic
gtcatcggcatgctcagtgccgtggtcgccagcatcatcgagtctattggtgactactacgcctgtgcacggctgtcc

SEQ ID NO: 4 - DNA, 118 bp, Artificial Sequence
  Synthetic
ttgcagcggcctcagctggcggggcctcctcccctgctttcgccacccggcgcctcgcccctcgaccccgcggcagctgggcccgggcgctctgcttccctcggccctttgtggctct

SEQ ID NO: 5 - DNA, 70 bp, Artificial Sequence
  Synthetic
cctcacgctctttgcagtctatgcgtccaacgtgctcttttgcgcgctgcccaacatgggtctcggtagc

SEQ ID NO: 6 - DNA, 73 bp, Artificial Sequence
  Synthetic
agcactgcgctgcgacctagttttccctttggaatcaggtcctctctcctgcgtttacattggcctctcccac

SEQ ID NO: 7 - DNA, 68 bp, Artificial Sequence
  Synthetic
ccacatcacgaggcaagaaggaaatggggccgtcggtccccgcagaaccactcatcgccgggctagag

SEQ ID NO: 8 - DNA, 69 bp, Artificial Sequence
  Synthetic
ccagtgtaacacccagcgctgctgattggctcccgtctcggctctgggtcgcctggacaccgtgattgg

SEQ ID NO: 9 - DNA, 25 bp, Artificial Sequence
  Synthetic
acgcttatcttccgatacctctatt

SEQ ID NO: 10 - DNA, 26 bp, Artificial Sequence
  Synthetic
tgtcgttagaagtaggcgttatagtg

SEQ ID NO: 11 - DNA, 24 bp, Artificial Sequence
  Synthetic
gctaatcacaacctaacgcctaat

SEQ ID NO: 12 - DNA, 26 bp, Artificial Sequence
  Synthetic
gtatttgtttgtacgtcggtggtagt

SEQ ID NO: 13 - DNA, 27 bp, Artificial Sequence
  Synthetic
ggatagtcgtgtataggcgtagtagtt

SEQ ID NO: 14 - DNA, 27 bp, Artificial Sequence
  Synthetic
atcatcgacatactcaataccgtaatc

SEQ ID NO: 15 - DNA, 25 bp, Artificial Sequence
  Synthetic
ttacaacgacctcaactaacgaaac

SEQ ID NO: 16 - DNA, 26 bp, Artificial Sequence
  Synthetic
agagttataaagggtcgagggaagta

SEQ ID NO: 17 - DNA, 25 bp, Artificial Sequence
  Synthetic
cctcacgctctttacaatctatacg

SEQ ID NO: 18 - DNA, 25 bp, Artificial Sequence
  Synthetic
gttatcgagatttatgttgggtagc

SEQ ID NO: 19 - DNA, 25 bp, Artificial Sequence
  Synthetic
aacactacgctacgacctaattttc

SEQ ID NO: 20 - DNA, 25 bp, Artificial Sequence
  Synthetic
gtgggagaggttaatgtaaacgtag

SEQ ID NO: 21 - DNA, 27 bp, Artificial Sequence
  Synthetic
ctctaacccgacgataaataattctac

SEQ ID NO: 22 - DNA, 27 bp, Artificial Sequence
  Synthetic
ttatattacgaggtaagaaggaaatgg

SEQ ID NO: 23 - DNA, 25 bp, Artificial Sequence
  Synthetic
ttaattacggtgtttaggcgattta

SEQ ID NO: 24 - DNA, 25 bp, Artificial Sequence
  Synthetic
ccaatataacacccaacgctactaa

SEQ ID NO: 25 - DNA, 30 bp, Artificial Sequence
  Synthetic
ggatttattaggtgtgtaatgttatggatt

SEQ ID NO: 26 - DNA, 31 bp, Artificial Sequence
  Synthetic
actccacataaatcttctaaactaaattcct

SEQ ID NO: 27 - DNA, 22 bp, Artificial Sequence
  Synthetic
tttgtatgtggtgggagggttt

SEQ ID NO: 28 - DNA, 23 bp, Artificial Sequence
  Synthetic
acaaaaaaacacaccactcccaa

SEQ ID NO: 29 - DNA, 28 bp, Artificial Sequence
  Synthetic
gtagttttagatggttttttgagttgga

SEQ ID NO: 30 - DNA, 26 bp, Artificial Sequence
  Synthetic
cactcccatcctatcttccctctata

SEQ ID NO: 31 - DNA, 31 bp, Artificial Sequence
  Synthetic
gaggtaaatgtaggtttttagtgttgatttt

SEQ ID NO: 32 - DNA, 33 bp, Artificial Sequence
  Synthetic
ctttatcctcccaatactaattattatttctcc

SEQ ID NO: 33 - DNA, 28 bp, Artificial Sequence
  Synthetic
gagtgtttgttagttttagaaatagagg

SEQ ID NO: 34 - DNA, 22 bp, Artificial Sequence
  Synthetic
ggtaactccactccaacttaca

SEQ ID NO: 35 - DNA, 23 bp, Artificial Sequence
  Synthetic
ttgggaagggaggggatattagg

SEQ ID NO: 36 - DNA, 26 bp, Artificial Sequence
  Synthetic
ccccctctctttcaacttaaataaac

SEQ ID NO: 37 - DNA, 20 bp, Artificial Sequence
  Synthetic
ggggtggggtataggatagt

SEQ ID NO: 38 - DNA, 24 bp, Artificial Sequence
  Synthetic
tcccacaattcaataaaaactacc

SEQ ID NO: 39 - DNA, 30 bp, Artificial Sequence
  Synthetic
aattggaaaataaattagagttataaaggg

SEQ ID NO: 40 - DNA, 20 bp, Artificial Sequence
  Synthetic
aaacctcctcccctactttc

SEQ ID NO: 41 - DNA, 21 bp, Artificial Sequence
  Synthetic
ggtaggtgtttgggtattgta

SEQ ID NO: 42 - DNA, 25 bp, Artificial Sequence
  Synthetic
ggatacctatttctacaaccactac

SEQ ID NO: 43 - DNA, 25 bp, Artificial Sequence
  Synthetic
gagagaggatttgattttaaaggga

SEQ ID NO: 44 - DNA, 29 bp, Artificial Sequence
  Synthetic
tccaaatacattcactaaaacatttccaa

SEQ ID NO: 45 - DNA, 20 bp, Artificial Sequence
  Synthetic
aggtaagaaggaaatggggt

SEQ ID NO: 46 - DNA, 28 bp, Artificial Sequence
  Synthetic
aactataaacctaaaccctaaaactccc

SEQ ID NO: 47 - DNA, 22 bp, Artificial Sequence
  Synthetic
ggtttttattgggttagtttgg

SEQ ID NO: 48 - DNA, 19 bp, Artificial Sequence
  Synthetic
taaccacccaaaccctacc

SEQ ID NO: 49 - DNA, 20 bp, Artificial Sequence
  Synthetic
gagtaggtagagtcggggac

SEQ ID NO: 50 - DNA, 27 bp, Artificial Sequence
  Synthetic
acttctacaactaaattcgaaaacgtc

SEQ ID NO: 51 - DNA, 22 bp, Artificial Sequence
  Synthetic
ttttgggcgtagagtagcggtt

SEQ ID NO: 52 - DNA, 25 bp, Artificial Sequence
  Synthetic
ctctaaatctcgataaaactcgcat

SEQ ID NO: 53 - DNA, 19 bp, Artificial Sequence
  Synthetic
acgcgcgtcggaggatttc

SEQ ID NO: 54 - DNA, 20 bp, Artificial Sequence
  Synthetic
aatcgccacccgaacgaacg

SEQ ID NO: 55 - DNA, 24 bp, Artificial Sequence
  Synthetic
ttgtaggttagggagattacgttt

SEQ ID NO: 56 - DNA, 24 bp, Artificial Sequence
  Synthetic
ctatctctatatctctatctcccg

SEQ ID NO: 57 - DNA, 26 bp, Artificial Sequence
  Synthetic
gcggtttagtatcggtgggagatcgt

SEQ ID NO: 58 - DNA, 25 bp, Artificial Sequence
  Synthetic
taaaccaaaatcgaaaatcgcgacc

SEQ ID NO: 59 - DNA, 26 bp, Artificial Sequence
  Synthetic
ttacgtcggaggaggtattaacgaga

SEQ ID NO: 60 - DNA, 32 bp, Artificial Sequence
  Synthetic
cttaaaaatctacccacaacaacatcgaaacg

SEQ ID NO: 61 - DNA, 20 bp, Artificial Sequence
  Synthetic
tttttttagatacgtgcggt

SEQ ID NO: 62 - DNA, 30 bp, Artificial Sequence
  Synthetic
cctctctactaactaaacccctttatacta

SEQ ID NO: 63 - DNA, 26 bp, Artificial Sequence
  Synthetic
tcggttaattaatttgggaggcgaaa

SEQ ID NO: 64 - DNA, 24 bp, Artificial Sequence
  Synthetic
gacgcgatacgactcactccgcta

SEQ ID NO: 65 - DNA, 21 bp, Artificial Sequence
  Synthetic
gtagtttcggtagaggcgttt

SEQ ID NO: 66 - DNA, 26 bp, Artificial Sequence
  Synthetic
ctactaaattactaaacgaatcgaaa

SEQ ID NO: 67 - DNA, 24 bp, Artificial Sequence
  Synthetic
ggtttaaaattcgagaaaataacg

SEQ ID NO: 68 - DNA, 20 bp, Artificial Sequence
  Synthetic
acgcctcaaaaactccgaaa

SEQ ID NO: 69 - DNA, 32 bp, Artificial Sequence
  Synthetic
gagttatagtgagtcggttacgtaaatagcga

SEQ ID NO: 70 - DNA, 30 bp, Artificial Sequence
  Synthetic
attacttatcaacgccgacaaactaccgct

SEQ ID NO: 71 - DNA, 24 bp, Artificial Sequence
  Synthetic
ttacgttattggttggagggtgcg

SEQ ID NO: 72 - DNA, 20 bp, Artificial Sequence
  Synthetic
ctaactaaaccgcgcgaacg

SEQ ID NO: 73 - DNA, 32 bp, Artificial Sequence
  Synthetic
agagttatagtgagtcggttacgtaaatagcg

SEQ ID NO: 74 - DNA, 32 bp, Artificial Sequence
  Synthetic
cttattacttatcaacgccgacaaactaccgc

SEQ ID NO: 75 - DNA, 25 bp, Artificial Sequence
  Synthetic
gtagttgagttgtaggatgtaagcg

SEQ ID NO: 76 - DNA, 20 bp, Artificial Sequence
  Synthetic
ccaaaccctactaaaacccg

SEQ ID NO: 77 - DNA, 21 bp, Artificial Sequence
  Synthetic
tttaaggtaggggattttcgg

SEQ ID NO: 78 - DNA, 29 bp, Artificial Sequence
  Synthetic
aaaatcctatcaaccctttaaaactcgaa

SEQ ID NO: 79 - DNA, 21 bp, Artificial Sequence
  Synthetic
cgtaggtaggtgaaagtaggc

SEQ ID NO: 80 - DNA, 21 bp, Artificial Sequence
  Synthetic
gaacttcctctctattccccc

SEQ ID NO: 81 - DNA, 23 bp, Artificial Sequence
  Synthetic
ttatagaaattaggaggcgcgta

SEQ ID NO: 82 - DNA, 20 bp, Artificial Sequence
  Synthetic
gcaactcctctaaaccgaac

SEQ ID NO: 83 - DNA, 24 bp, Artificial Sequence
  Synthetic
agatatttttaaaaggtagcgaaa

SEQ ID NO: 84 - DNA, 25 bp, Artificial Sequence
  Synthetic
acactacctctccaacataaaaacg

SEQ ID NO: 85 - DNA, 23 bp, Artificial Sequence
  Synthetic
agttataacgtattgaagttgcg

SEQ ID NO: 86 - DNA, 26 bp, Artificial Sequence
  Synthetic
ttattataaacgctcaaatrgaaaaa

SEQ ID NO: 87 - DNA, 20 bp, Artificial Sequence
  Synthetic
gtaggggtagggattacggt

SEQ ID NO: 88 - DNA, 30 bp, Artificial Sequence
  Synthetic
ttaatctcctaaacaacaatatataaccga

SEQ ID NO: 89 - DNA, 20 bp, Artificial Sequence
  Synthetic
gttttgagtcgtacgcgttg

SEQ ID NO: 90 - DNA, 22 bp, Artificial Sequence
  Synthetic
cttaaaaaccaaaatcctccga

SEQ ID NO: 91 - DNA, 20 bp, Artificial Sequence
  Synthetic
tagtagcgcggagttggttt

SEQ ID NO: 92 - DNA, 20 bp, Artificial Sequence
  Synthetic
actactaccaccgctaccgc

SEQ ID NO: 93 - DNA, 20 bp, Artificial Sequence
  Synthetic
taaagggaatgtggggtttc

SEQ ID NO: 94 - DNA, 24 bp, Artificial Sequence
  Synthetic
cgcttccttctcttaaactaaaaa

SEQ ID NO: 95 - DNA, 22 bp, Artificial Sequence
  Synthetic
agaatttcgtttagggtagggc

SEQ ID NO: 96 - DNA, 25 bp, Artificial Sequence
  Synthetic
gatacgaatttctaatacgctaacc

SEQ ID NO: 97 - DNA, 21 bp, Artificial Sequence
  Synthetic
atttagattgttagtagcggg

SEQ ID NO: 98 - DNA, 22 bp, Artificial Sequence
  Synthetic
aaaacacaaaccgaaacaaaac

SEQ ID NO: 99 - DNA, 26 bp, Artificial Sequence
  Synthetic
tataataaggtgatgtgttaggaagc

SEQ ID NO: 100 - DNA, 22 bp, Artificial Sequence
  Synthetic
aaaacaaacaaaaacaaacgta

SEQ ID NO: 101 - DNA, 20 bp, Artificial Sequence
  Synthetic
aaagaggggagagagttcgc

SEQ ID NO: 102 - DNA, 20 bp, Artificial Sequence
  Synthetic
ctatatcctcgaccccgatt

SEQ ID NO: 103 - DNA, 25 bp, Artificial Sequence
  Synthetic
agtaatcgtagggtagtggacgggg

SEQ ID NO: 104 - DNA, 29 bp, Artificial Sequence
  Synthetic
ctctaaaacgaactacccttaaacgcgcc

SEQ ID NO: 105 - DNA, 22 bp, Artificial Sequence
  Synthetic
aggtacgtgatgaaattttggt

SEQ ID NO: 106 - DNA, 24 bp, Artificial Sequence
  Synthetic
gaaatttaacgcccaatataaacc

SEQ ID NO: 107 - DNA, 20 bp, Artificial Sequence
  Synthetic
tcgtggaaggaagtacgttt

SEQ ID NO: 108 - DNA, 26 bp, Artificial Sequence
  Synthetic
cgacataaaattcaactaaataaccg

SEQ ID NO: 109 - DNA, 20 bp, Artificial Sequence
  Synthetic
gaattagagcgattcgggac

SEQ ID NO: 110 - DNA, 22 bp, Artificial Sequence
  Synthetic
gtcaaaactcccgcttcatatt

SEQ ID NO: 111 - DNA, 26 bp, Artificial Sequence
  Synthetic
tagtgagtagagaaagacgtacgaaa

SEQ ID NO: 112 - DNA, 21 bp, Artificial Sequence
  Synthetic
gcctatctacaccctatcgcc

(19) United States
(12) Patent Application Publication
(10) Pub. No.: US 2024/0240244 A1
(43) Pub. Date: Jul. 18, 2024

(54) Methylation Detection Method and Kit for Genomic DNA or Cell-Free DNA Molecules in Biological Sample

(71) Applicant: AnchorDx Medical Co., Ltd., Guangzhou (CN)

(72) Inventors: Zhiwei Chen, Guangzhou (CN); Linhao Xu, Guangzhou (CN); Jun Wang, Guangzhou (CN); Xin Liu, Guangzhou (CN); Zhujia Ye, Guangzhou (CN); Jianbing Fan, Guangzhou (CN)

(21) Appl. No.: 18/012,618

(22) PCT Filed: Jun. 28, 2021
(86) PCT No.: PCT/CN2021/102727

§ 371 Date: Dec. 22, 2022

(30) Foreign Application Priority Data
Jun. 29, 2020 (CN) 202010609179.6
Jun. 9, 2021 (CN) 202110642369.2

(51) Int. Cl. C12Q 1/6858; C12Q 1/686

(52) U.S. Cl. CPC C12Q 1/6858; C12Q 1/686

(57) ABSTRACT

A methylation detection method and kit for genomic DNA or cell-free DNA molecules in a biological sample are provided. The method includes: obtaining a converted DNA molecule through a chemical and/or enzymatic reaction; performing a methylation-specific multiplex PCR, through multiple pairs of methylation-specific primers, on one or more methylated areas of one or more genes in the converted DNA molecule serving as a substrate, in order to obtain a PCR amplification product; and detecting the methylation levels of different fragments of the amplification product. The detection method can detect DNA methylation in multiple genes in multiple samples at the same time, requires only a small initial amount of DNA, is suitable for detecting methylated fragments having a small copy number, and has such advantages as high sensitivity, high specificity, high accuracy, a low detection limit, and low detection cost. The entire detection process and the subsequent data analysis step are simpler and more scientific than those of the prior art.

### TECHNICAL FIELD

The present invention pertains to the technical field of genetic tests. More particularly, the invention relates to a methylation detection method and kit for genomic DNA or cell-free DNA molecules in a biological sample.

### DESCRIPTION OF RELATED ART

Gene expression can be regulated by DNA methylation. The detection of DNA methylation, therefore, can at least provide some clues as to the regulation of gene expression. Moreover, it has been proved that DNA methylation is involved in a series of important biological processes including early embryonic development, genomic imprinting, inactivation of the X chromosome, the silencing of a repeated sequence, and the development, progression, and metastasis of cancer. Research results have also shown that one major application of DNA methylation is to serve as a biomarker for the early screening and prognosis of tumors.

Humans have approximately 3 billion base pairs, and DNA methylation takes place mainly in the CpG dinucleotides. The human genome has 28 million or so CpG sites, about 60% to 80% of which are methylated. While certain gene areas such as the promoter areas have a sequence rich in CpG sites (also known as a CpG island), CpG islands are seldom methylated. When it comes to tumor cells, however, the overall methylation level is lowered to 20% to 50%, meaning the methylation states of 10% to 60% of the CpG sites in the original normal cells, i.e., about 2.8 to 16.8 million CpG sites, may have changed, especially in the cancer suppressor genes.

The existing DNA methylation detection methods mainly include whole-genome DNA methylation detection and site-specific methylation detection. Whole-genome DNA methylation detection is used primarily in research, to find differential methylated sites, and so on, can be categorized as high-throughput-sequencing-based or chip-based whole-genome DNA methylation detection. Site-specific methylation detection is used chiefly in translational medicine and includes methylation-specific polymerase chain reaction (PCR) (or MSP for short), bisulfite sequencing PCR (BSP), pyrosequencing, and mass spectrometry.

In an actual clinical application or operation, the detection substrate generally has a low DNA content. In early cancer screening, for example, the percentage of circulating tumor DNA (ctDNA) in the circulating free DNA (cfDNA) in a plasma sample is relatively low (can be as low as 0.01%). The concentration of cfDNA in a healthy person's plasma sample ranges approximately from 0 to 100 ng/mL, with the average concentration being about 30 ng/mL, and the length of a ctDNA fragment is generally about 166 bp (when the fragment is produced by lysis of a dead cell) or 150-250 bp (when the fragment is secreted from an extracellular vesicle released by a cancer cell). Under such circumstances, those conventional methods involving capturing DNA through hybridization and performing high-throughput sequencing on the captured DNA have low sensitivity and specificity. As for whole-genome methylation sequencing, a complex process flow is entailed, and subsequent analysis is complicated, not to mention the relatively high capture cost. PCR-based methods such as BSP are capable of detecting methylation but have two deficiencies due to the lack of CpG sites in the primers used. First, as it is not allowed for the primers to include CpG sites, and C is generally converted into T by bisulfite sequencing (BS), the actual operation is relatively difficult, especially in primer design in CpG islands, and the difficult operation is compounded by such issues as low GC contents, low PCR efficiency, and an excessive amount of non-specific product. Second, the signals of a trace amount of methylated fragments tend to be overwhelmed by the signals of unmethylated fragments because of the lack of selectivity of PCR. For example, if BSP is used to detect a sequence that, in 99 out of 100 cases, may not be methylated when secreted from a normal cell but, in 1 out of 100 cases, is methylated when secreted from a cancer cell, the 1% information may get lost when the other 99% cannot be identified as noise or real signals. The use of MSP or a chip can cover only a limited number of CpG sites (the typical target being a single site) and hence has low flexibility. In cases where a common CpG island has a relatively large number of methylated sites, the conventional methods cannot detect the methylation states of the multiple CpG sites in such a sequence.

### BRIEF SUMMARY OF THE INVENTION

The present invention in one aspect provides a methylation detection method for genomic DNA or circulating free DNA molecules in a biological sample.

The technical solution to achieving the aforesaid objective includes the following:

A methylation detection method for genomic DNA or circulating free DNA molecules in a biological sample includes:
obtaining a converted DNA molecule through a chemical and/or enzymatic reaction;
performing a methylation-specific, i.e., methylation-dependent, multiplex polymerase chain reaction (PCR), through multiple pairs of methylation-specific primers, on one or more methylated areas of one or more genes in the converted DNA molecule serving as a substrate, in order to obtain a PCR amplification product; and
detecting the methylation levels of different fragments of the amplification product.

The detection method of the present invention can detect DNA methylation in multiple genes in multiple samples at the same time. The entire detection process and the subsequent data analysis step are simple and scientific and require only a small amount of DNA to start with, making the detection method suitable for detecting methylated fragments having a small copy number. The detection method advantageously features high sensitivity, high specificity, a low detection limit, and low detection cost.

The present invention in another aspect provides a methylation detection kit for genomic DNA or circulating free DNA molecules in a biological sample.

A DNA methylation detection kit includes PCR primers specific to at least one sequence selected from the group consisting of SEQ ID NO.1-SEQ ID NO.8, wherein the primers are:
a forward primer of SEQ ID NO.9 and a reverse primer of SEQ ID NO.10, both serving as PCR primers specific to SEQ ID NO.1; and/or
a forward primer of SEQ ID NO.11 and a reverse primer of SEQ ID NO.12, both serving as PCR primers specific to SEQ ID NO.2; and/or
a forward primer of SEQ ID NO.13 and a reverse primer of SEQ ID NO.14, both serving as PCR primers specific to SEQ ID NO.3; and/or
a forward primer of SEQ ID NO.15 and a reverse primer of SEQ ID NO.16, both serving as PCR primers specific to SEQ ID NO.4; and/or
a forward primer of SEQ ID NO.17 and a reverse primer of SEQ ID NO.18, both serving as PCR primers specific to SEQ ID NO.5; and/or
a forward primer of SEQ ID NO.19 and a reverse primer of SEQ ID NO.20, both serving as PCR primers specific to SEQ ID NO.6; and/or
a forward primer of SEQ ID NO.21 and a reverse primer of SEQ ID NO.22, both serving as PCR primers specific to SEQ ID NO.7; and/or
a forward primer of SEQ ID NO.23 and a reverse primer of SEQ ID NO.24, both serving as PCR primers specific to SEQ ID NO.8.

The detection method of the present invention uses a methylation-dependent multiplex PCR technique to amplify and enrich a methylated area of interest rapidly and specifically, and the methylation-dependent multiplex PCR technique is optimized as follows: a high-fidelity polymerase is used to perform methylation-dependent multiplex PCR amplification, and the multiplex PCR product is processed with an endonuclease or DNA-fragment-sorting magnetic beads and then purified. The inventor has found that the foregoing optimization is effective in removing non-specific product in the methylation-dependent multiplex PCR amplification product and can therefore prevent the non-specific product from affecting subsequent detection (e.g., from generating noise and occupying the sequencing depth during the detection process), increase the accuracy of the detection result effectively, and lower the detection limit. In particular, the specificity of the methylation-dependent multiplex PCR in the invention can be enhanced by performing the methylation-dependent multiplex PCR with a Phusion U DNA polymerase. The detection method of the invention also uses suitable reference sequences to normalize the detection results of samples with different methylation ratios so as to enable identification of the differences in methylation and further increase the accuracy and reliability of the detection results.

The present invention uses a methylation-dependent multiplex PCR technique and optimizes both the methylation-dependent multiplex PCR amplification method and the product processing method in order to overcome such prior art drawbacks as the existing methylation detection methods requiring a large amount of DNA to start with and the conventional multiplex PCR amplification products not being directly suitable for high-throughput detection. The invention has high sensitivity, high specificity, high accuracy, a low detection limit, and low detection cost as its advantages, can detect a methylation level as low as 0.05%, has a simpler and more scientific detection process and data analysis step than the prior art, and is particularly suitable for detecting methylated fragments having a small copy number.

### BRIEF DESCRIPTION OF THE SEVERAL VIEWS OF THE DRAWINGS

FIG. 1 schematically shows the process flow of the detection method of the present invention.

FIG. 2 shows the information about the two exemplary CpG sites in the sequence of SEQ ID NO.5 in embodiment 3.

FIG. 3 is a plot showing the limit of quantification (LOQ) detection results, obtained by the detection method of the invention, of the two exemplary CpG sites in the sequence of SEQ ID NO.5 in embodiment 3.

FIG. 4 is plots showing the LOQ detection results, obtained by bisulfite sequencing PCR, of the same two CpG sites in the sequence of SEQ ID NO.5 in embodiment 3.

FIG. 5 is charts showing the detection results, obtained respectively by the detection method of the invention and bisulfite sequencing PCR, of CpG site 1 (ch5_40681550) in the samples in embodiment 4.

FIG. 6 is charts showing the detection results, obtained respectively by the detection method of the invention and bisulfite sequencing PCR, of CpG site 2 (ch5_40681569) in the samples in embodiment 4.

FIG. 7 is a chart showing the detection results of the dimer percentages of products obtained by methylation-dependent multiplex PCR amplification performed respectively with a Q5U enzyme and a Phusion U enzyme.

FIG. 8 is a chart showing the detection results of the dimer percentages and target sequencing percentages of products obtained by methylation-dependent multiplex PCR amplification performed respectively by a conventional PCR method and a touchdown PCR method.

FIG. 9 is a chart showing the detection results of the dimer percentages of methylation-dependent multiplex PCR amplification products purified respectively with XP Beads and Smart Beads.

FIG. 10 is a chart showing the detection results of the dimer percentages and target sequencing percentages of methylation-dependent multiplex PCR amplification products purified respectively with XP Beads and by column purification.

FIG. 11 is a chart comparing the detection results corresponding respectively to methylation-dependent multiplex PCR amplification with different numbers of multiplex PCR amplification cycles.

FIG. 12 shows satisfactory linear regression of signals detected from the standards in embodiment 5, wherein the standards include 17 target sites and have different methylation concentrations respectively.

FIG. 13 shows the receiver operating characteristic (ROC) curve of embodiment 6.

FIG. 14 shows the ROC curve of embodiment 7.

### DETAILED DESCRIPTION OF THE INVENTION

In cases where the conditions of an experiment method used in any of the following embodiments of the present invention are not specified, the conventional conditions (e.g., the conditions stated in Sambrook et al., Molecular Cloning: Cold Spring Harbor Laboratory Press, New York, 1989) or the conditions suggested by the related manufacturer(s) generally apply. All the common chemical reagents used in the embodiments are commercially available.

Unless otherwise defined, each technical or scientific term used in relation to the present invention has the same connotation as generally understood by a person skilled in the art. All such terms used in this specification serve only to expound the embodiments but not to limit the invention.

The terms “comprise” and “have” and their variations are intended to cover non-exclusive inclusions. For example, a process, method, device, product, or apparatus comprising a series of steps or modules is not limited to the specified steps or modules and may further include, in an optional manner, steps or modules that are not specified or are inherent to the process, method, device, product, or apparatus.

As used herein, the terms “a plurality of” and “multiple” refer to two or more than two. The term “and/or” indicates three possible relationships between the objects connected by the term; for example, “A and/or B” may indicate that A exists alone, that A and B coexist, or that B exists alone. The symbol “/”′ generally indicates “or”.

As used herein, the term “converted DNA molecule” may refer to either of the following: 1) A DNA molecule obtained by converting the cytosines (unmethylated) in the DNA molecule into uracils through a deamination reaction while preserving the 5′-methylcytosines in the DNA molecule, wherein the conversion reagent used in some embodiments may be either sodium bisulfite or a ten-eleven translocation (TET) enzyme plus an apolipoprotein B mRNA editing enzyme, catalytic polypeptide (APOBEC) enzyme (an enzymatic method). 2) A DNA molecule obtained by converting the 5′-methylcytosines in the DNA molecule into dihydrouracil by a mixed chemical-enzymatic method while preserving the unmethylated cytosine in the DNA molecule, wherein the conversion reagent used in some embodiments may be a TET enzyme plus pyridine borane.

As used herein, the term “methylation-specific primer” refers to a PCR primer designed for a methylated area. Such a primer can bind specifically to a methylated area in a converted DNA molecule.

As used herein, the term “methylated area” refers to a specific genomic area that includes a CpG site.

As used herein, the term “methylation-specific multiplex PCR”, also known as “methylation-dependent multiplex PCR”, refers to PCR amplification of multiple target methylated sites in the same PCR amplification system and concurrently of the adjacent 40-10,000 bp areas by way of methylation-specific primers, wherein the target methylated sites may be located in the primers or the amplicons.

As used herein, the term “methylation level” may refer to: 1) the methylation ratio of a specific CpG site; 2) the average of the methylation ratios of multiple CpG sites in a specific region; or 3) the normalized read of a methylation-amplified fragment.

As used herein, the term “fragment” refers to a fragment whose size ranges from 40 to 10,000 bp.

As used herein, the term “sequencing” includes second-generation sequencing and third-generation sequencing. The principle of second-generation sequencing lies in massive parallel sequencing (MPS). In some embodiments, second-generation sequencing may be: 1) sequencing by synthesis (SBS), i.e., sequencing by DNA polymerase-based synthesis, some representative companies of which are Illumina (reversible terminator sequencing), Thermo Fisher/Life Technologies (Ion Torrent), GenapSys, and Roche Diagnostics (454 pyrosequencing); or 2) sequencing by ligation (SBL), i.e., sequencing by DNA ligase-based ligation, some representative companies of which are BGI Genomics/Complete Genomics (combinatorial probe-anchor ligation (cPAL)) and Thermo Fisher/Applied Biosystems (sequencing by oligonucleotide ligation and detection (SOLID). Third-generation sequencing is single-molecule sequencing. In some embodiments, third-generation sequencing may be: single-molecule real-time fluorescent sequencing (SMRT) (Pacific Biosciences), nanopore sequencing (Oxford Nanopore Technologies (ONT), and Genia Technologies and Stratos Genomics (Roche Diagnostics)), nanogate sequencing (Quantum Biosystems), or sequencing by DNA hydrolysis (e.g., de-synthesis or pyrophosphorolysis) (Base4).

As used herein, the term “universal array” refers to a technique that entails fixing one of a known target DNA fragment (which may be a methylated fragment) and an unknown nucleic acid sequence to a specific vector in an ordered array, allowing sequence-complementary hybridization to take place, and using a fluorescent technique and pattern recognition analysis to detect the hybridization result. In some embodiments, the universal array may be: 1) the microarray technique, some representative companies of which are Affymetrix (Thermo Fisher)/Agilent; 2) the bead array technique, one representative company of which is Illumina; 3) the xMAP (Multi-Analyte Profiling) technique, one representative company of which is Luminex; or 4) the nCounter technique, one representative company of which is NanoString.

Some embodiments of the present invention are related to a methylation detection method for genomic DNA or cell-free DNA molecules in a biological sample, and the method includes the following steps:
obtaining a converted DNA molecule through a chemical and/or enzymatic reaction; performing a methylation-specific, i.e., methylation-dependent, multiplex PCR, through multiple pairs of methylation-specific primers, on one or more methylated areas of one or more genes in the converted DNA molecule serving as a substrate, in order to obtain a PCR amplification product; and
detecting the methylation levels of different fragments of the amplification product.

In some embodiments, the method more specifically includes the following steps:
S1. extracting genomic DNA or circulating free DNA from the biological sample, processing the extracted DNA with the chemical reaction to obtain the converted DNA molecule, and performing the methylation-specific, i.e., methylation-dependent, multiplex PCR on the converted DNA molecule through the methylation-specific primers, wherein different methylation-specific PCR primers are used on different genes or different methylated sites;
S2. processing and purification of the multiplex PCR product: processing the multiplex PCR product obtained from step S1 with an endonuclease or DNA-fragment-sorting magnetic beads in order to obtain a processed product, and purifying the processed product in order to obtain a purified product; and
S3. detecting the methylation levels of the purified product obtained from step S2.

In some embodiments, the biological sample includes a tissue sample, a cell sample, or a fluid sample of an organism and is preferably selected from the group consisting of blood, blood serum, blood plasma, a vitreous body, sputum, urine, tear, sweat, and saliva.

In some embodiments, the genomic DNA or circulating free DNA extracted from the biological sample is subjected to chemical conversion.

In some embodiments, the chemical reaction used for DNA conversion includes a chemical method (e.g., a bisulfite method), an enzymatic method (e.g., through a TET enzyme or an APOBEC enzyme), or a mixed chemical-enzymatic method (e.g., through a TET enzyme and pyridine borane).

In some embodiments, the chemical method and/or the enzymatic method converts the unmethylated cytosine in a DNA molecule into uracil and preserves the 5′-methylcytosine in the DNA molecule in order to obtain the converted DNA molecule.

In some embodiments, the mixed chemical-enzymatic method converts the 5′-methylcytosine in a DNA molecule into dihydrouracil and preserves the unmethylated cytosine in the DNA molecule in order to obtain the converted DNA molecule.

In some embodiments, the method further includes processing and purifying the multiplex PCR product by: processing the obtained multiplex PCR product with an endonuclease or DNA-fragment-sorting magnetic beads in order to obtain a processed product, and purifying the processed product.

In some embodiments, the method more specifically includes the following steps:
S1. extracting genomic DNA or circulating free DNA (also known as cell-free DNA) from the sample, processing the extracted DNA with a bisulfite, and performing the methylation-dependent multiplex PCR on the processed DNA through the specific primers, wherein different specific PCR primers are used on different genes or different methylated sites;
S2. processing and purification of the multiplex PCR product: processing the multiplex PCR product obtained from step S1 with an endonuclease or DNA-fragment-sorting magnetic beads in order to obtain a processed product, and purifying the processed product in order to obtain a purified product; and
S3. detecting the methylation levels of the purified product obtained from step S2.

Here, the term “methylation-dependent multiplex PCR” refers to PCR amplification of multiple methylated sites of interest in the same PCR amplification system and concurrently of the adjacent 50-300 bp areas, wherein the methylated sites of interest may be located in the primers or the amplicons.

Here, the term “specific PCR primer” refers to a primer designed for a methylated site of interest or an adjacent CpG site. Each specific PCR primer includes a methylated CpG site that has been successfully converted with sodium bisulfite so as to ensure specific amplification of the methylated DNA.

The detection method of the present invention can detect the methylation levels of multiple genes in multiple samples at the same time. Different specific PCR primers are used for different genes or different methylated sites, and during the detection process, different specific tag sequences are used for different samples in order to distinguish the samples from one another.

In some embodiments, the reaction conditions of the methylation-dependent multiplex PCR in step S1 are: 98° C. for 30 s to 5 min; followed by 10-25 cycles each starting with 98° C. for 15 s and continuing with 60±10° C. for 15 s to 10 min and then with 68° C.-72° C. for 15 s to 5 min; followed by 68° C.-72° C. for 0 min to 15 min.

In some embodiments, the reaction conditions of the methylation-dependent multiplex PCR in step S1 are: 98° C. for 30 s to 5 min; followed by 10-25 cycles each starting with 98° C. for 15 s and continuing with 60±5° C. for 15 s to 10 min and then with 72° C. for 15 s to 5 min; followed by 72° C. for 0 min to 15 min.

In some embodiments, the reaction conditions of the methylation-dependent multiplex PCR in step S1 are: 98° C. for 30 s to 5 min; followed by 10-25 cycles each starting with 98° C. for 15 s and continuing with 60±5° C. for 15 s to 5 min and then with 72° C. for 15 s to 5 min; followed by 72° C. for 0 min to 15 min.

In some embodiments, the amplification method of the methylation-dependent multiplex PCR in step S1 is a touchdown PCR method. The inventor has found through research that, in the detection system of the present invention, performing the methylation-dependent multiplex PCR by a touchdown PCR method can effectively increase the specificity of the methylation-dependent multiplex PCR and thereby reduce the product of non-specific amplification.

In some embodiments, the reaction conditions of the methylation-dependent multiplex PCR in step S1 are: 98° C. for 30 s; followed by 5-10 cycles each starting with 98° C. for 15 s and continuing with 65±3° C. (or a temperature successively lowered therefrom by 0.2-0.8° C. after each cycle) for 15 s and then with 72° C. for 15 s; followed by 10-25 cycles each starting with 98° ° C. for 15 s and continuing with 60±10° C. for 15 s to 10 min and then with 68° C.-72° C. for 15 s to 5 min; followed by 72° ° C. for 0 min to 15 min.

In some embodiments, the reaction conditions of the methylation-dependent multiplex PCR in step S1 are: 98° C. for 30 s; followed by 5-10 cycles each starting with 98° C. for 15 s and continuing with 65±3° C. (or a temperature successively lowered therefrom by 0.2-0.8° C. after each cycle) for 15 s and then with 72° C. for 15 s; followed by 10-25 cycles each starting with 98° C. for 15 s and continuing with 60±5° C. for 15 s to 10 min and then with 68-72° C. for 15 s to 5 min; followed by 72° C. for 0 min to 15 min.

In some embodiments, the reaction conditions of the methylation-dependent multiplex PCR in step S1 are: 98° C. for 30 s; followed by 5-10 cycles each starting with 98° C. for 15 s and continuing with 65±3° C. (or a temperature successively lowered therefrom by 0.2-0.8° C. after each cycle) for 15 s and then with 72° C. for 15 s; followed by 10-25 cycles each starting with 98° C. for 15 s and continuing with 60±5° C. for 15 s to 5 min and then with 68-72° C. for 15 s to 5 min; followed by 72° C. for 0 min to 15 min.

In some embodiments, the reaction conditions of the methylation-dependent multiplex PCR in step S1 are: 98° C. for 30 s; followed by 5-10 cycles each starting with 98° C. for 15 s and continuing with 65±3° C. (or a temperature successively lowered therefrom by 0.2° ° C.-0.8° ° C. after each cycle) for 15 s and then with 72° C. for 15 s; followed by 15-20 cycles each starting with 98° C. for 15 s and continuing with 60±3° C. for 15 s and then with 72° C. for 15 s; followed by 72° C. for 15 min.

In some embodiments, the reaction conditions of the methylation-dependent multiplex PCR in step S1 are preferably: 98° C. for 30 s; followed by 10 cycles each starting with 98° C. for 15 s and continuing with 65±3° C. (or a temperature successively lowered therefrom by 0.5° C. after each cycle) for 15 s and then with 72° C. for 15 s; followed by 15 cycles each starting with 98° C. for 15 s and continuing with 60±3° ° C. for 15 s and then with 72° C. for 15 s; followed by 72° C. for 15 min. The inventor has found through research that the most target genes can be successfully detected when the multiplex PCR amplification is carried out under these reaction conditions.

In some embodiments, the methylation-dependent multiplex PCR in step S1 uses an enzyme selected from the group consisting of a Phusion DNA polymerase, a Q5 enzyme, a Hieff enzyme, a KAPA DNA polymerase, a Pfu enzyme, a SuperFi enzyme, and a corresponding uracil-compatible polymerase, preferably from the group consisting of a Phusion U enzyme, a KAPA U enzyme, a Q5U enzyme, and a HieffU enzyme.

In some embodiments, the enzyme used in the methylation-dependent multiplex PCR in step S1 is a Phusion DNA polymerase.

In some embodiments, the enzyme used in the methylation-dependent multiplex PCR is preferably the Phusion U Hot Start DNA polymerase. The Phusion U Hot Start DNA polymerase can better increase the specificity of the methylation-dependent multiplex PCR in the detection method of the present invention and thereby reduce the product of non-specific amplification.

In some embodiments, the touchdown PCR method is a nested PCR method.

In some embodiments, the methylation-dependent multiplex PCR in step S1 is performed on one or more different genes and/or one or more different reference sequences simultaneously.

In some embodiments, the one or more different reference sequences are at least one selected from the group consisting of:
reference sequence 1: a sequence in the EPHA3 gene that corresponds to the fragment obtained by PCR amplification with SEQ ID NO.25 and SEQ ID NO.26 serving as specific PCR primers;
reference sequence 2: a sequence in the KBTBD4 gene that corresponds to the fragment obtained by PCR amplification with SEQ ID NO.27 and SEQ ID NO.28 serving as specific PCR primers;
reference sequence 3: a sequence in the PLEKHF1 gene that corresponds to the fragment obtained by PCR amplification with SEQ ID NO.29 and SEQ ID NO.30 serving as specific PCR primers; and
reference sequence 4: a sequence in the SYT10 gene that corresponds to the fragment obtained by PCR amplification with SEQ ID NO.31 and SEQ ID NO.32 serving as specific PCR primers.

In some embodiments, the one or more different reference sequences are at least two selected from the group consisting of reference sequences 1-4. Using at least two of the reference sequences contributes to more consistent detection results and higher accuracy in comparing the detection results of different samples.

In some embodiments, the one or more different genes are at least one selected from the group consisting of SEQ ID NO.1-SEQ ID NO.8.

In some embodiments, the methylation-dependent multiplex PCR is performed on at least one sequence selected from the group consisting of SEQ ID NO.1-SEQ ID NO.8 under the following reaction conditions: 98° C. for 30 s; followed by 10 cycles each starting with 98° C. for 15 s and continuing with 65±3° C. (or a temperature successively lowered therefrom by 0.2-0.8° C. after each cycle) for 15 s and then with 72° C. for 15 s; followed by 15-20 cycles each starting with 98° ° C. for 15 s and continuing with 60±3° C. for 15 s and then with 72° C. for 15 s; followed by 72° C. for 15 min.

In some embodiments, the methylation-dependent multiplex PCR is performed on at least one sequence selected from the group consisting of SEQ ID NO.1-SEQ ID NO.8 under the following reaction conditions: 98° C. for 30 s; followed by 10 cycles each starting with 98° C. for 15 s and continuing with 65±3° C. (or a temperature successively lowered therefrom by 0.5° C. after each cycle) for 15 s and then with 72° C. for 15 s; followed by 15 cycles each starting with 98° C. for 15 s and continuing with 60±3° ° C. for 15 s and then with 72° C. for 15 s; followed by 72° C. for 15 min.

In some embodiments, the processing with the endonuclease in step S2 is carried out at 37±1° C. for 10 min to 15 min, with the final concentration of the endonuclease in the processing system being 1-10 U/μL.

In some embodiments, the processing with the endonuclease in step S2 is carried out at 37±1° C. for 10 min to 15 min, with the final concentration of the endonuclease in the processing system being 3.5-4.5 U/μL.

In some embodiments, the processing with the endonuclease in step S2 is preferably carried out at 37±1° C. for 10 min, with the final concentration of the endonuclease in the processing system being 4 U/μL.

In some embodiments, the endonuclease is a T4 endonuclease.

In some embodiments, the DNA-fragment-sorting magnetic beads in step S2 are XP magnetic beads.

In some embodiments, the purification in step S2 is purification with magnetic beads.

In some embodiments, the magnetic beads used for the magnetic-bead-based purification are XP magnetic beads. The inventor has found through research that using XP magnetic beads in the detection method of the present invention leads to better purification results.

In some embodiments, the methylation levels of the purified product obtained from step S2 are detected in step S3 by a sequencing method.

In some embodiments, the sequencing method includes second-generation sequencing and third-generation sequencing. Second-generation sequencing is based on massive parallel sequencing (MPS) and may be, for example, sequencing by synthesis (SBS) or sequencing by ligation (SBL). Third-generation sequencing is single-molecule sequencing and may be, for example, single-molecule real-time fluorescent sequencing, nanopore sequencing, nanogate sequencing, or sequencing by DNA hydrolysis.

In some embodiments, the sequencing method in step S3 includes the following steps: (1) repairing, and simultaneously adding a base A to, the 3′ end of the purified product obtained from step S2; (2) ligating a sequencing adapter to the product obtained from step (1); (3) performing PCR amplification, through an adapter primer, on the ligation product obtained from step (2) in order to obtain a sequencing library; and (4) pooling, according to the same mole number, libraries prepared from different samples, and sequencing the pooled libraries.

In some embodiments, step (3) includes 3-7 PCR amplification cycles. Preferably, step (3) includes 7 PCR amplification cycles.

In some embodiments, step (3) further includes purifying, and performing quality inspection on, the sequencing library.

In some embodiments, the sequencing library is purified with XP magnetic beads.

In some embodiments, the sequencing method includes second-generation sequencing and third-generation sequencing. Second-generation sequencing is based on massive parallel sequencing (MPS). Third-generation sequencing is single-molecule sequencing.

In some embodiments, the second-generation sequencing may be sequencing by synthesis (SBS) or sequencing by ligation (SBL).

In some embodiments, the third-generation sequencing may be single-molecule real-time fluorescent sequencing, nanopore sequencing, nanogate sequencing, or sequencing by DNA hydrolysis.

In the present invention, the sequencing method in step S3, i.e., the multi-sample multi-fragment DNA methylation detection method used to detect the methylation levels of the purified product obtained from step S2, is named Methylation-Dependent Amplification and Sequencing (MeDAS).

In some embodiments, a universal array method is used in step S3 to detect the methylation levels of the purified product obtained from step S2. The universal array refers to a technique that entails fixing one of a known target DNA fragment (which may be a methylated fragment) and an unknown nucleic acid sequence to a specific vector in an ordered array, allowing sequence-complementary hybridization to take place, and using a fluorescent technique and pattern recognition analysis to detect the hybridization result. During the detection process, different specific tag sequences are used for different samples in order to distinguish the samples from one another.

In some embodiments, the universal array technique may be the microarray technique, the bead array technique, the xMAP (Multi-Analyte Profiling) technique, or the nCounter technique.

In some embodiments, the sample is a body fluid, cell, or tissue of an organism.

In some embodiments, the body fluid of an organism is a body fluid secreted by an organ or tissue of the human body, and the human body may be in a normal or pathological state.

In some embodiments, the body fluid of an organism is blood, urine, saliva, sweat, cerebrospinal fluid, fluid in the pleural cavity, or ascitic fluid.

In some embodiments, the body fluid of an organism is preferably blood serum, blood plasma, a vitreous body, sputum, urine, tear, sweat, or saliva.

Another embodiment of the present invention provides a DNA methylation detection kit including PCR primers specific to at least one sequence selected from the group consisting of SEQ ID NO.1-SEQ ID NO.8, wherein the primers are:
a forward primer of SEQ ID NO.9 and a reverse primer of SEQ ID NO.10, both serving as PCR primers specific to SEQ ID NO.1; and/or
a forward primer of SEQ ID NO.11 and a reverse primer of SEQ ID NO.12, both serving as PCR primers specific to SEQ ID NO.2; and/or
a forward primer of SEQ ID NO.13 and a reverse primer of SEQ ID NO.14, both serving as PCR primers specific to SEQ ID NO.3; and/or
a forward primer of SEQ ID NO.15 and a reverse primer of SEQ ID NO.16, both serving as PCR primers specific to SEQ ID NO.4; and/or
a forward primer of SEQ ID NO.17 and a reverse primer of SEQ ID NO.18, both serving as PCR primers specific to SEQ ID NO.5; and/or
a forward primer of SEQ ID NO.19 and a reverse primer of SEQ ID NO.20, both serving as PCR primers specific to SEQ ID NO.6; and/or
a forward primer of SEQ ID NO.21 and a reverse primer of SEQ ID NO.22, both serving as PCR primers specific to SEQ ID NO.7; and/or
a forward primer of SEQ ID NO.23 and a reverse primer of SEQ ID NO.24, both serving as PCR primers specific to SEQ ID NO.8.

In some embodiments, the kit further includes at least one of the following four groups of specific PCR primers:
specific PCR primers composed of a forward primer of SEQ ID NO.25 and a reverse primer of SEQ ID NO.26;
specific PCR primers composed of a forward primer of SEQ ID NO.27 and a reverse primer of SEQ ID NO.28;
specific PCR primers composed of a forward primer of SEQ ID NO.29 and a reverse primer of SEQ ID NO.30; and
specific PCR primers composed of a forward primer of SEQ ID NO.31 and a reverse primer of SEQ ID NO.32.

In some embodiments, the kit includes at least two of the foregoing four groups of specific PCR primers.

In some embodiments, the kit includes the foregoing four groups of specific PCR primers.

In some embodiments, the kit further includes a Phusion DNA polymerase, a T4 endonuclease, and/or DNA-purifying magnetic beads.

In some embodiments, the Phusion DNA polymerase is preferably the Phusion U Hot Start DNA polymerase.

In some embodiments, the DNA-purifying magnetic beads are preferably XP magnetic beads.

In some embodiments, the kit further includes a sequencing library construction reagent.

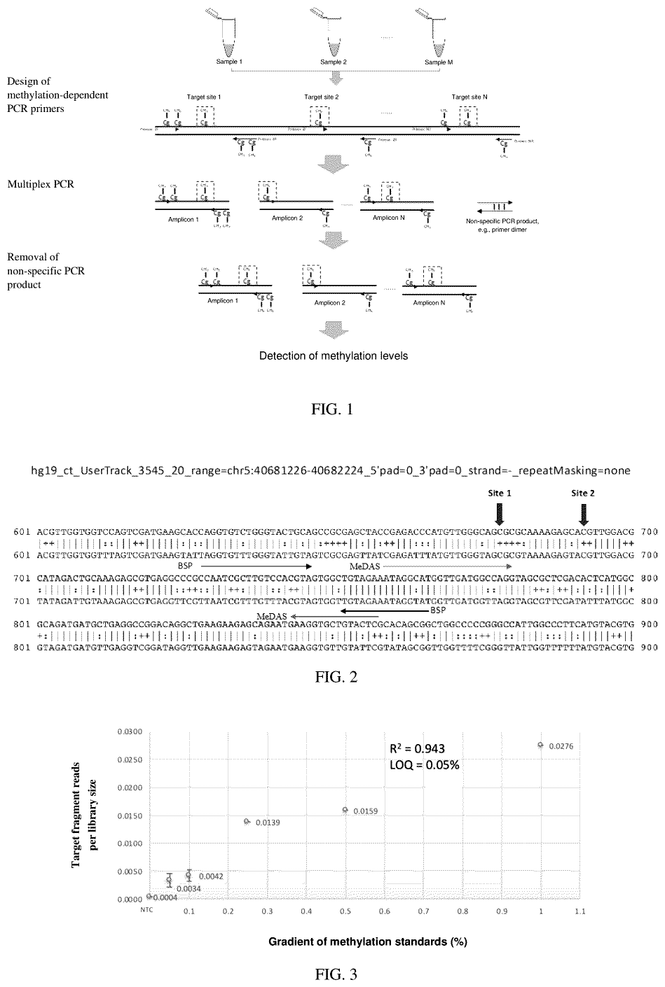

The detection process flow of the detection method provided by the present invention is shown in FIG. 1.

### Embodiment 1

In this embodiment, a multi-sample multi-fragment DNA methylation detection method includes the following steps:

S1. extracting genomic DNA or circulating free DNA from a sample, processing the extracted DNA with the EZDNA Methylation-Gold (ZYMO) kit, and performing methylation-dependent multiplex PCR on the processed DNA through specific primers, wherein the enzyme used in the PCR is a high-fidelity polymerase selected from the group consisting of a Phusion DNA polymerase (e.g., the Phusion Hot Start II DNA Polymerase or the Phusion U Hot Start DNA Polymerase), a Q5 enzyme (e.g., the Q5® Hot Start High-Fidelity DNA Polymerase or the Q5U® Hot Start High-Fidelity DNA Polymerase), a Hieff enzyme (e.g., the Hieff NGS® HG Hot Start Multiplex PCR Enzyme), a KAPA DNA polymerase (e.g., the KAPA HiFi Uracil+ Kit or the KAPA2G Fast Hot Start DNA Polymerase), a Pfu enzyme, and a SuperFi enzyme; wherein the Phusion U Hot Start DNA Polymerase is preferably used in this embodiment; wherein a touchdown PCR method is used as the PCR amplification method; and wherein different specific PCR primers are used for different genes or different methylated sites;

S2. processing and purification of the multiplex PCR product either by applicable method 1, which includes processing the multiplex PCR product obtained from step S1 with a T4 endonuclease, the processing conditions being 37±1° C. for 10-15 min, with the final enzyme concentration in the processing system being 1-10 U/μL (preferably, the processing conditions being 37±1° C. for 10 min, with the final enzyme concentration in the processing system being 4 U/μL), and purifying the enzymatically processed PCR product, the purification method being purification with XP magnetic beads; or by applicable method 2, which includes processing and purifying the multiplex PCR product directly with DNA-fragment-sorting magnetic beads, wherein the magnetic beads are XP magnetic beads; and

S3. detecting, by a sequencing method, the methylation levels of the purified product obtained from step S2: (1) repairing, and simultaneously adding a base A to, the 3′ end of the purified product obtained from step S2; (2) ligating a sequencing adapter to the product obtained from step (1); (3) performing PCR amplification, through an adapter primer, on the ligation product obtained from step (2), with the number of PCR cycles being 3-7 (preferably 7), then purifying the amplification product in order to obtain a sequencing library, and performing quality inspection on and quantifying the sequencing library obtained; and (4) pooling, according to the same mole number, libraries prepared from different samples, and sequencing the pooled libraries with the Illumina MiSeq/MiSeqDx/NextSeq/NextSeqDx platform.

The detection method provided by this embodiment is named Methylation-Dependent Amplification and Sequencing (MeDAS) and can be used to perform DNA methylation detection on multiple genes in multiple samples at the same time. The entire detection process and the subsequent data analysis step are simple and scientific and require only a small initial amount of DNA, making the detection method suitable for detecting methylated fragments having a small copy number. Moreover, the detection method advantageously features high sensitivity, high specificity, a low detection limit, and low detection cost. TABLE 1 compares the features of the MeDAS method of the present invention with those of some existing methylation detection methods:

TABLE 1
Comparison between the MeDAS method of the present invention and some existing methylation detection methods

| Feature | MeDAS | MSP | MethyLight | BSP |
|---|---|---|---|---|
| Number of target | Moderate - large - very large (dozens | Small (<10 | Small (<10 | Small - moderate - large (several to |
| detection areas | to more than one thousand target | target areas) | target areas) | more than one hundred target areas) |
| (in amplicon) | areas), with a flexible range | | | |
| Sample throughput | Low - medium - high (several to | Low (1-30) | Low (1-30) | Low - medium (several to dozens) |
| | hundreds, or even more than one | | | |
| | thousand), with a flexible range | | | |
| Detection | NGS | DNA gel | qPCR | Sanger sequencing or NGS |
| equipment | | electrophoresis | | |
| Sequencing depth | Small - moderate (the coverage being | No | No | High (the coverage being thousands of |
| | hundreds of times to more than one | | | times to more than ten thousand times) |
| | thousand times) | | | |
| Detection signal | Reads of amplicons and sequence | DNA band | Fluorescent | Reads of amplicons (both methylated |
| | information of the target areas | | signal | and unmethylated) |
| Internal reference | Yes or no (depending on the | No | Yes | No |
| sequence (reference | normalization method) | | | |
| sequence) | | | | |
| What is | 1) Relative methylation ratios of the | Whether the target | Relative | Methylation ratios of the target areas |
| determined | target areas; | areas are methylated | methylation | |
| | 2) methylation haplotype information | | ratios of the | |
| | of the target areas | | target areas | |
| Detection property | Semi-quantitative | Qualitative | Semi-quantitative | Quantitative |
| Expenses (including | Low - medium (dozens to more than one | Low (dozens of | Low (dozens of | High (hundreds of RMB per sample) |
| only expenses for | hundred RMB per sample), related to | RMB per sample) | RMB per sample) | |
| reagents and | the number of target areas and sample | | | |
| sequencing, not | throughput | | | |
| including expenses | | | | |
| for sample | | | | |
| collection and | | | | |
| processing in the | | | | |
| initial stage) | | | | |

In the table above, MeDAS stands for Methylation-Dependent Amplification and Sequencing, MSP stands for Methylation-Specific PCR, and BSP stands for Bisulfite Sequencing PCR.

It can be known from TABLE 1 that the MeDAS technique provided by the present invention has such advantages over the prior art as high throughput, high sensitivity, high specificity, a low detection limit, and low detection cost.

### Embodiment 2

In this embodiment, a DNA methylation detection kit includes reagents for performing methylation level detection on the sequences of SEQ ID NO.1-SEQ ID NO.8. The sequences of SEQ ID NO.1-SEQ ID NO.8 are shown in TABLE 2:

TABLE 2
Sequences of SEQ ID NO. 1-SEQ ID NO. 8

| SEQ ID | | Genomic position (hg19 | |
|---|---|---|---|
| NO. | Code | reference genome) | Sequence (5′-3′) |
| 1 | ZY-104 | Chr7_ | TGCCGCTAGAAGCAGGCGTCACAGTGCCC |
| | | 50347009-50347085(−) | CCAGAGTGCTTGTCAGTCTTAGAAACAGAG |
| | | | GCATCGGAAGACAAGCGT |
| 2 | ZY-131 | Chr2_ | GCTAGTCACAGCCTGGCGCCTGGTGTCCCC |
| | | 177029539-177029622(+) | TCCCTTCCCAAGCCCCCTCAGCTTTTCCACT |
| | | | GCCACCGGCGTACAAGCAAGTGC |
| 3 | ZY-137 | Chr20_ | GTCATCGGCATGCTCAGTGCCGTGGTCGCC |
| | | 4850587-4850664(−) | AGCATCATCGAGTCTATTGGTGACTACTAC |
| | | | GCCTGTGCACGGCTGTCC |
| 4 | ZY-148 | Chr1_ | TTGCAGCGGCCTCAGCTGGCGGGGCCTCCT |
| | | 157948715-157948832(+) | CCCCTGCTTTCGCCACCCGGCGCCTCGCCC |
| | | | CTCGACCCCGCGGCAGCTGGGCCCGGGCG |
| | | | CTCTGCTTCCCTCGGCCCTTTGTGGCTCT |
| 5 | ZY-154 | Chr5_ | CCTCACGCTCTTTGCAGTCTATGCGTCCAA |
| | | 40681506-40681575(+) | CGTGCTCTTTTGCGCGCTGCCCAACATGGG |
| | | | TCTCGGTAGC |
| 6 | ZY-62 | Chr5_ | AGCACTGCGCTGCGACCTAGTTTTCCCTTT |
| | | 90653216-90653288(+) | GGAATCAGGTCCTCTCTCCTGCGTTTACAT |
| | | | TGGCCTCTCCCAC |
| 7 | ZY-7 | Chr1_ | CCACATCACGAGGCAAGAAGGAAATGGGG |
| | | 63795438-63795505(−) | CCGTCGGTCCCCGCAGAACCACTCATCGCC |
| | | | GGGCTAGAG |
| 8 | ZY-8 | Chr12_ | CCAGTGTAACACCCAGCGCTGCTGATTGGC |
| | | 133000021-133000089(−) | TCCCGTCTCGGCTCTGGGTCGCCTGGACAC |
| | | | CGTGATTGG |

More specifically, the kit includes the following constituents:

(1) PCR primers specific to the sequences of SEQ ID NO.1-SEQ ID NO.8, as shown in TABLE 3:

TABLE 3
PCR primers specific to the sequences of SEQ ID NO. 1-SEQ ID NO. 8

| To-be- | | | | |
|---|---|---|---|---|
| detected | | | | |
| SEQ ID | | SEQ ID | | SEQ ID |
| NO. | Forward primer (5′-3′) | NO. | Reverse primer (5′-3′) | NO. |
| 1 | ACGCTTATCTTCCGATACC | 9 | TGTCGTTAGAAGTAGGCGT | 10 |
| | TCTATT | | TATAGTG | |
| 2 | GCTAATCACAACCTAACGC | 11 | GTATTTGTTTGTACGTCGG | 12 |
| | CTAAT | | TGGTAGT | |
| 3 | GGATAGTCGTGTATAGGCG | 13 | ATCATCGACATACTCAATA | 14 |
| | TAGTAGTT | | CCGTAATC | |
| 4 | TTACAACGACCTCAACTAA | 15 | AGAGTTATAAAGGGTCGA | 16 |
| | CGAAAC | | GGGAAGTA | |
| 5 | CCTCACGCTCTTTACAATC | 17 | GTTATCGAGATTTATGTTG | 18 |
| | TATACG | | GGTAGC | |
| 6 | AACACTACGCTACGACCTA | 19 | GTGGGAGAGGTTAATGTA | 20 |
| | ATTTTC | | AACGTAG | |
| 7 | CTCTAACCCGACGATAAAT | 21 | TTATATTACGAGGTAAGAA | 22 |
| | AATTCTAC | | GGAAATGG | |
| 8 | TTAATTACGGTGTTTAGGC | 23 | CCAATATAACACCCAACGC | 24 |
| | GATTTA | | TACTAA | |

(2) PCR primers specific to reference sequences 1-4 (Ref1-4):
reference sequence 1: a sequence in the EPHA3 gene that corresponds to the fragment obtained by PCR amplification with SEQ ID NO.25 and SEQ ID NO.26 serving as specific PCR primers;
reference sequence 2: a sequence in the KBTBD4 gene that corresponds to the fragment obtained by PCR amplification with SEQ ID NO.27 and SEQ ID NO.28 serving as specific PCR primers;
reference sequence 3: a sequence in the PLEKHF1 gene that corresponds to the fragment obtained by PCR amplification with SEQ ID NO.29 and SEQ ID NO.30 serving as specific PCR primers; and
reference sequence 4: a sequence in the SYT10 gene that corresponds to the fragment obtained by PCR amplification with SEQ ID NO.31 and SEQ ID NO.32 serving as specific PCR primers.

The details of the reference sequences are summarized in TABLE 4:

TABLE 4
Information about PCR primers specific to reference sequences 1-4

| Reference | Corresponding | | Forward primer (5′- | SEQ ID | Reverse primer | SEQ |
|---|---|---|---|---|---|---|
| sequence | gene name | Gene code | 3′) | NO. | (5′-3′) | ID NO. |
| Ref1 | EPHA3 | NM_005233 | GGATTTATTAGG | 25 | ACTCCACAT | 26 |
| | | | TGTGTAATGTTAT | | AAATCTTCTA | |
| | | | GGATT | | AACTAAATT | |
| | | | | | CCT | |
| Ref2 | KBTBD4 | NM_001318724 | TTTGTATGTGGTG | 27 | ACAAAAAAA | 28 |
| | | | GGAGGGTTT | | CACACCACT | |
| | | | | | CCCAA | |
| Ref3 | PLEKHF1 | NM_024310 | GTAGTTTTAGAT | 29 | CACTCCCATC | 30 |
| | | | GGTTTTTTGAGTT | | CTATCTTCCC | |
| | | | GGA | | TCTATA | |
| Ref4 | SYT10 | NM_198992 | GAGGTAAATGTA | 31 | CTTTATCCTC | 32 |
| | | | GGTTTTTAGTGTT | | CCAATACTA | |
| | | | GATTTT | | ATTATTATTT | |
| | | | | | CTCC | |

(3) The Phusion U Hot Start DNA Polymerase, the T4 endonuclease, and the Agencourt AMPure XP Beads.

The PCR primers provided in the kit as specific to the sequences of SEQ ID NO.1-SEQ ID NO.8 were designed and successfully obtained by the inventor after an extensive research and analysis. Those detection primers can ensure the sensitivity and specificity of methylation-dependent multiplex PCR amplification but have no effect on the depth of sequent sequencing. The sequencing depth required for detecting methylated fragments under the detection limit of 2 copies to 0.05% methylation is less than that of an existing method (bisulfite sequencing PCR) by about 2-3 orders of magnitude.

The reference sequences 1-4 (Ref1-4) provided in the kit were obtained through optimization by the inventor, are consistently expressed in different samples, are not subject to the methylation level, and are suitable for use to normalize sequencing data so that the DNA methylation levels of different samples can be compared with one another to produce accurate and reliable comparison results.

### Embodiment 3

In this embodiment, detection results obtained with the DNA methylation detection method of the present invention (i.e., the MeDAS method) were compared with those obtained with bisulfite sequencing PCR (BSP). More specifically, the methylation states of the sequences of SEQ ID NO.1-SEQ ID NO.8 as stated in relation to embodiment 2 were detected separately with the DNA methylation detection method of the invention and BSP. The DNA methylation detection method of the invention used the kit in embodiment 2 and was carried out as follows:

S1. DNA Standards were Obtained and then Processed with a Bisulfite. After that, Methylation-Dependent Multiplex PCR Amplification was Performed on the Processed DNA Standards.

(1) Preparation of a cfDNA mock standard

1) Genomic DNA quantification with Qubit HS

a) Preparation of a Qubit solution:

TABLE 5
Formula of Qubit solution

| Ingredient | Volume (μL) |
|---|---|
| Qubit dsDNA HS Buffer (QB) | 199 |
| Qubit dsDNA HS Reagent (QA) | 1 |
| Total volume | 200 |

b) Dilution of the genomic DNA sample: 1 μL of DNA sample+9 μL of EB (elution buffer) were measured out. The DNA sample was diluted 10 times, before 1 μL of the diluted

DNA was quantified.

2) Shearing of the genomic DNA

The genomic DNA was diluted with EB to about 10 ng/μL, and then 300 μL of the diluted DNA was measured out. The three shearing tubes accompanying the kit (each tube having a specification of 130 μL) were each added with 50 μL of the diluted DNA (repeated twice) in order to carry out the following shearing procedure:

TABLE 6
cfDNA shearing procedure

| | | | Cycles | | |
|---|---|---|---|---|---|
| Peak incident | Duty | Treatment | per | Temperature | Sample |
| power (w) | factor | time(s) | burst | (° C.) | volume (μL) |
| 50 | 40% | 240 | 200 | 20 | 50 |

3 μL of the sheared DNA was taken out and kept at 4° ° C., waiting for its concentration to be determined and its quality to be inspected with 2100 Bioanalyzer.

3) Purification and storage of the sheared DNA

a) Preparation of 80% ethanol

b) Purification steps: allowing undiluted AMPure magnetic beads to reach equilibrium at room temperature for 30 min→measuring out 240 μL of the magnetic beads, mixing with all the sheared genomic DNA sufficiently in a 1.5 mL LoBind tube with a vortex mixer, and subjecting the mixture to simple centrifugation→incubation at room temperature for 5 min→placing on the magnetic base for 3-4 min, or until the solution becomes clear→transferring the supernatant to a new 1.5 mL LoBind tube→adding 240 μl of undiluted AMPure magnetic beads into the tube→mixing sufficiently with the vortex mixer, followed by simple centrifugation→incubation at room temperature for 5 min→placing on the magnetic base for 4 min, or until the solution becomes clear, followed by removal of the supernatant by suction→adding 1.5 mL of the 80% ethanol in order to carry out washing for 30 s, at the end of which the supernatant is removed→adding 1.5 mL of the 80% ethanol in order to carry out washing for 30 s, at the end of which the supernatant is removed→centrifugation for 1 min, followed by placement on the magnetic base until the liquid becomes clear, followed by removal of excessive liquid by suction→opening the lid and allowing the contents of the tube to dry until the magnetic bead surface is no longer reflective→adding 100 μL of EB for elution, followed by a thorough mix, simple centrifugation, and incubation at room temperature for 2 min→placing on the magnetic base for magnetic attraction for 2 min, or until the liquid becomes clear, followed by collecting the supernatant and transferring the supernatant to a new 1.5 mL LoBind tube; and then repeating the elution step once, with the final volume of the DNA sample being 200 μL.

c) Storage: Storage at −20° C.

4) Quantification and quality inspection of the purified DNA

Quality inspection: 1 μL of the purified cfDNA mock sample was measured out, and the quality of the sample was inspected with 2100 Bioanalyzer. Quantification: 1 μL of the purified sample was measured out and was quantified with Qubit HS.

(2) Preparation of DNA standard substrates with a concentration gradient by a mixing process

1) Preparation of a 100%-methylation control by processing the genomic DNA with a bisulfite

The HG DNA and the cfDNA mock were processed with the EZDNA Methylation-Gold (ZYMO) kit as follows:

a) Preparation of associated buffers:

a. Preparation of the CT Conversion Reagent: 900 μL of water, 50 μL of M-Dissolving Buffer, and 300 μL of M-Dilution Buffer were added into a tube of CT Conversion Reagent in order for the reagent to be dissolved at room temperature. The tube was then placed on a shaker and shaken for 10 min.

b. Preparation of the M-WASH BUFFER: 24 mL of absolute ethanol was added into a flask of M-WASH BUFFER, and the lid of the flask was marked.

b) Adding the following into a PCR tube:

a. 500 ng of DNA, followed by water until the total volume reached 20 μL, wherein the volume of the water added was calculated according to the desired concentration; and

b. 130 μL of the CT Conversion Reagent. The test tube or pipet was then gently tapped to mix the sample.

c) The sample tube was placed in a thermal cycler, which was operated as follows: setting the thermal cycler at 98° ° C. for 10 min and then at 64° C. for 14 h.

d) The column of a collection tube was put into the collection tube, and 600 μL of M-Binding Buffer was added into the column, followed by the sample in step c The column was then covered with its lid and inverted several times to mix the sample.

e) Centrifugation at full speed (>10,000×g) for 30 s, followed by removal of the waste liquid.

f) 200 μL of M-Wash Buffer was added into the column, and then centrifugation was performed at full speed for 30 s.

g) 200 μL of M-Desphonation Buffer was added into the column, and the sample was allowed to rest at room temperature (20-30° C.) for 15-20 min.

h) Centrifugation at full speed for 30 s, followed by removal of the waste liquid.

i) 200 μL of M-Wash Buffer was added into the column, and then centrifugation was performed at full speed for 30 s.

j) 200 μL more of M-Wash Buffer was added, and then centrifugation was performed for 30 s.

k) 25 μL of M-Elution Buffer was added directly to the substrate in the column, and the column was placed into a 1.5 mL tube. DNA elution was carried out by full-speed centrifugation to obtain the final product, i.e., a converted product (which when kept at −20° C. remained good for use for 1 week).

1) The degrees of methylation conversion at 7-10 CpG, CHG, and CHH sites in the standard were determined by a sequencing analysis.

2) Preparation of standards with a concentration gradient of 0%, 0.05%, 0.1%, 0.25%, 0.5%, 1%, and 100% by mixing the 0%-methylation control with the 100%-methylation control

(3) The methylation-dependent multiplex PCR of the present invention and multiplex bisulfide-specific PCR (multiplex BSP) were separately performed on the sequences of SEQ ID NO.1-SEQ ID NO.8 as stated in relation to embodiment 2 and on reference sequences 1-4. The methylation-dependent multiplex PCR primers for use with the sequences of SEQ ID NO.1-SEQ ID NO.8 have been shown above in relation to embodiment 2, and so have the primers for use with reference sequences 1-4. The multiplex BSP primers for use with the sequences of SEQ ID NO.1-SEQ ID NO.8 are shown in TABLE 7.

TABLE 7
Multiplex BSP primers

| To-be- | | | | |
|---|---|---|---|---|
| detected | | SEQ | | SEQ |
| SEQ ID | Forward primer | ID | Reverse primer | ID |
| NO. | (5′-3′) | NO. | (5′-3′) | NO. |
| 1 | GAGTGTTTGTTAGTTT | 33 | GGTAACTCCACTCCAA | 34 |
| | TAGAAATAGAGG | | CTTACA | |
| 2 | TTGGGAAGGGAGGGGA | 35 | CCCCCTCTCTTTCAAC | 36 |
| | TATTAGG | | TTAAATAAAC | |
| 3 | GGGGTGGGGTATAGGA | 37 | TCCCACAATTCAATAA | 38 |
| | TAGT | | AAACTACC | |
| 4 | AATTGGAAAATAAATT | 39 | AAACCTCCTCCCCTAC | 40 |
| | AGAGTTATAAAGGG | | TTTC | |
| 5 | GGTAGGTGTTTGGGTA | 41 | GGATACCTATTTCTAC | 42 |
| | TTGTA | | AACCACTAC | |
| 6 | GAGAGAGGATTTGATT | 43 | TCCAAATACATTCACT | 44 |
| | TTAAAGGGA | | AAAACATTTCCAA | |
| 7 | AGGTAAGAAGGAAATG | 45 | AACTATAAACCTAAAC | 46 |
| | GGGT | | CCTAAAACTCCC | |
| 8 | GGTTTTTATTGGGTTA | 47 | TAACCACCCAAACCCT | 48 |
| | GTTTGG | | ACC | |

The methylation-dependent multiplex PCR amplification system of the present invention is shown in TABLE 8, with the amplification procedure shown in TABLE 9:

TABLE 8
Methylation-dependent multiplex PCR amplification
system of the present invention

| | 1 RXN | | |
|---|---|---|---|
| Reagent | Volume (μL) | Final content | |
| 5X GC buffer | 10 | 1X | |
| 10 mM dNTP Mix | 2 | 400 | μM |
| Primer Set | — | 200 | nM each |
| MgCl₂ | 3 | 1.5 | mM |
| Phusion U Hot Start DNA Polymerase | 0.75 | 0.03 | U/μL |
| DNA | — | 10 | ng cfDNA |

| | | |
|---|---|---|
| H₂O | | |
| Final Volume | 50 | — |

TABLE 9
Methylation-dependent multiplex PCR amplification procedure
of the present invention (touchdown method)

| Temperature | | Time | | Number of cycles |
|---|---|---|---|---|
| 98° | C. | 30 | s | 1 |
| 98° | C. | 15 | s | 10 |
| 65° C. (lowered by 0.5° C. | | 15 | s | |
| after each cycle) | | | | |
| 72° | C. | 15 | s | |
| 98° | C. | 15 | s | 15 |
| 60° | C. | 15 | s | |
| 72° | C. | 15 | s | |
| 72° | C. | 5 | min | 1 |

| | | | |
|---|---|---|---|
| 4° | C. | For as long as needed | — |

The BSP detection system is shown in TABLE 10, with the detection procedure shown in TABLE 11:

TABLE 10
Multiplex BSP detection system

| | 1 RXN | | |
|---|---|---|---|
| Reagent | Volume (μL) | Final content | |
| 5X GC buffer | 10 | 1X | |
| 10 mM dNTP Mix | 2 | 400 | μM |
| Primer Set | — | 200 | nM each |
| MgCl₂ | 3 | 1.5 | mM |
| Phusion U Hot Start DNA Polymerase | 0.75 | 0.03 | U/μL |
| DNA | — | 10 | ng cfDNA |

| | | |
|---|---|---|
| H₂O | | |
| Final Volume | 50 | — |

TABLE 11
Multiplex BSP detection procedure

| | | |
|---|---|---|
| Temperature | Time | Number of cycles |

| | | | |
|---|---|---|---|
| 98° C. | 30 | s | 1 |
| 98° C. | 15 | s | 3 |
| 50° C. | 15 | s | |
| 72° C. | 15 | s | |
| 98° C. | 15 | s | 27 |
| 57° C. | 15 | s | |
| 72° C. | 15 | s | |
| 72° C. | 5 | min | 1 |

| | | |
|---|---|---|
| 4° C. | For as long as needed | — |

### S2. Processing and Purification of the PCR Products

(1) The foregoing methylation-dependent multiplex PCR products were processed with a T4 endonuclease, the processing conditions being 37±1° C. for 10 min, with the final T4 endonuclease concentration in the processing system being 4 U/μL.

(2) The T4 endonuclease-processed PCR product of each of the different samples was purified by Agencourt AMPure XP Beads as follows:

a) The AMPure XP Beads were allowed to reach equilibrium at room temperature for at least 30 min.

b) 50 ng of the DNA for which a library was to be constructed was added into a 1.5 mL centrifuge tube, followed by 90 μL of (1.8×) AMPure XP Beads. The tube was placed on a vortex mixer, where the sample was mixed at room temperature for 5 min.

c) The centrifuge tube was placed on the magnetic base until the solution became clear (about 3-5 min).

d) The supernatant in the centrifuge tube was carefully removed while the centrifuge tube was kept on the magnetic base.

e) 200 μL of 80% ethanol was added while the centrifuge tube remained on the magnetic base.

f) The tube was allowed to stand for 1 min in order for all the beads to settle. The ethanol was then removed, and e) and f) were repeated once.

g) Simple centrifugation was performed, and then the tube was placed on the magnetic base until the liquid became clear. Excessive liquid was removed by suction. The sample was then dried in a 37° C. heating module for 5 min, or until all the residual ethanol disappeared.

h) 55 μL of EB was added for elution, followed by a thorough mix and incubation at room temperature for 5 min.

i) Simple centrifugation was performed, and then the tube was placed on the magnetic base for 3-5 min, or until the liquid became clear. The supernatant was collected. (A total of 50 μL was collected. 2 μL was taken from each of a number of selected samples in order to carry out a Qubit test, and all such samples were subsequently replenished to 50 μL.)

S3. Detecting, by a Sequencing Method, the Methylation Levels of the Purified Products Obtained from Step S2

Library construction was carried out with the NEB library construction kit Z1901S/L, with specific tag sequences used to distinguish the different samples from one another. The library construction steps are as follows:

(1) The 3′ end of each purified product obtained from step S2 was repaired and was added with a base A at the same time.

An end-repairing and base A addition system was prepared in a 1.5 mL centrifuge tube according to the following table with the DNA purified product obtained from the previous step:

TABLE 12
End-repairing and base A addition system

| | Amount | | Total |
|---|---|---|---|
| | for each | Number | amount |
| Reaction system | mix (μL) | of mixes | (μL) |
| Product of previous step | 50 | Added once | |
| NEBNext Ultra II End Prep Enzyme | 3 | 101 | 303 |
| Mix | | | |
| NEBNext Ultra II End Prep Reaction | | | |
| Buffer | 7 | | 707 |
| Total volume | 10 | | 1010 |

Reaction process: 20° ° C. for 30 min→65° C. for 30 min→held at 4° C., with heated lid: 85° ° C., volume: 60 μL.

(2) A sequencing adapter was ligated to the product having its 3′ end added with the base A, in order to produce a sequencing-adapter-ligated product.

A ligation reaction system was prepared in a 1.5 mL centrifuge tube according to the following table with the product obtained from the previous step:

TABLE 13
Sequencing adapter ligation reaction system

| | Amount | | Total |
|---|---|---|---|
| | for each | Number | amount |
| Reaction system | mix (μL) | of mixes | (μL) |
| Product of previous step | 60 | Added once | |
| NEBNext Adaptor for Illumina | 2.5 | Added once | |
| NEBNext Ligation Enhancer | 1 | 100.5 | 100.5 |
| NEBNext Ultra II Ligation Master Mix | 30 | | 3015 |
| Total volume | 31 | | 3115.5 |

Note:
Non-diluted adaptor was used when input DNA > 100 ng; 1:10 diluted adaptor was used when 5 ng ≤ input DNA ≤ 100 ng; and 1:25 diluted adaptor was used when input DNA < 5 ng.

Reaction process: 20° ° C. for 15 min→held at 4° C., with heated lid closed, volume: 100 μL.

After the reaction, purification was conducted with Agencourt AMPure XP Beads. The DNA was then eluted with 20 μL of EB, and the supernatant (15 μL) was collected.

(3) The sequencing-adapter-ligated product was subjected to PCR amplification, with the number of PCR cycles being 7 in order to obtain a sequencing library. The reaction system is as follows:

TABLE 14
Indexing PCR system

| | Amount | |
|---|---|---|
| | for each | Number of |
| Reaction system | mix (μL) | mixes |
| Product of previous step | 15 | Added once |
| NEBNext Ultra II Q5 Master Mix | 25 | Added once |
| i5 index Primer | 5 | Added once |
| i7 index Primer | 5 | Added once |
| Total volume | 50 | |

Reaction process: 98° C. for 30 s→7 cycles (each starting with 98° C. for 10 s→65° C. for 75 s)→65° ° C. for 5 min→held at 4° C., with heated lid: 105° C., volume: 50 μL.

After the reaction, purification was conducted with Agencourt AMPure XP Beads. The DNA was then eluted with 35 μL of EB, and the supernatant (30 μL) was collected.

Quantification: The library obtained was quantified with Qubit® 2.0 (Invitrogen) and stored at −20° C.

Quality inspection with 2100 Bioanalyzer: Library concentration range for the 2100 Bioanalyzer inspection: 100 pg/μL-10 ng/μL. The inspection was performed according to the quick start guide “5.2100_HighSensitivityDNA_QSG”.

(4) Second-generation high-throughput sequencing

Libraries prepared from the different samples were pooled according to the same mole number. After that, second-generation high-throughput sequencing was performed, and the sequencing steps are as follows:

### 1) Sample Pooling

The mass concentration (ng/μL) of each library was measured with Qubit, and the average length (bp) of the libraries was determined with 2100 Bioanalyzer. Mass concentration was then converted into molar concentration by the following equation. To perform sequencing runs on the sequencing machine, the libraries were mixed, i.e., pooled, according to the ratios of their respective data amounts. After that, the theoretical molar concentrations of the pooled libraries were calculated.

$$\begin{matrix}Molar concentration \\ ( nM ) of library\end{matrix} = \frac{Mass concentration ( ng / μ L ) of library}{\begin{matrix}( 660 g / mol \times average length ( bp ) of ) \\ library ) \times 1000\end{matrix}} \times {10}^{6}$$

Note: The average length of libraries YG001-047 is 350 bp.

2) Single-end sequencing was performed on the samples according to the procedure of Illumina MiSeq PE-300. The sequencing results produced by MiSeq were DNA sequences in the fastq format. Each sequenced sequence was correlated to the corresponding sample through a sequencing library index and a sample tag sequence (also referred to as a barcode), before the methylation state of each CG site in the detected fragment of each sample was calculated.

The detection results are as follows:

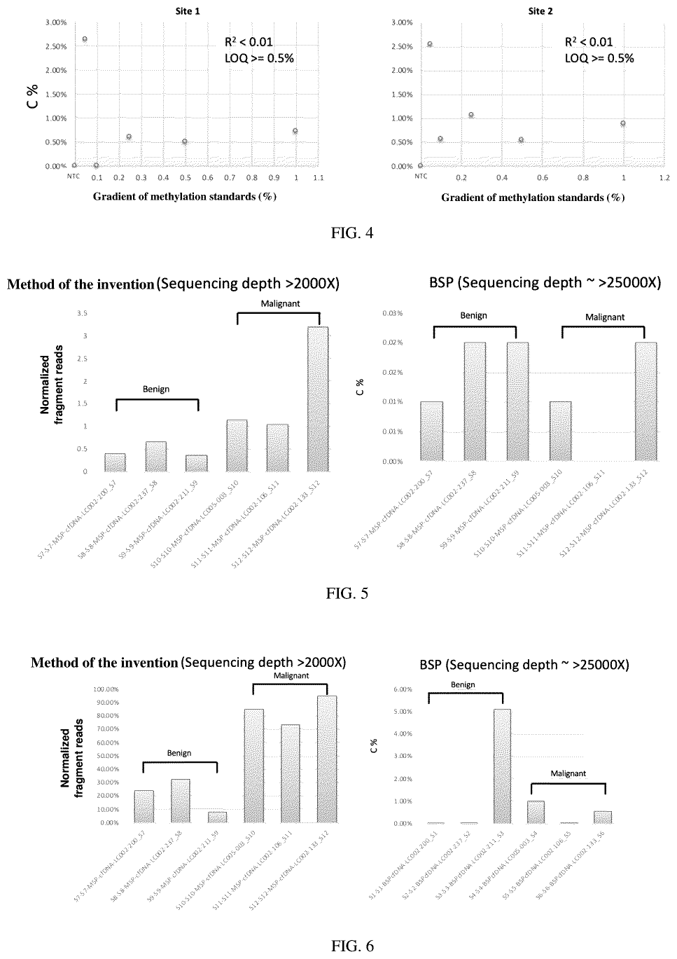

(1) The detection method of the present invention has a lower-LOQ hypothesis than bisulfite sequencing PCR: The detection method of the invention can detect 0.05% methylation (the average sequencing depth required was only 100-200×, and there was a linear trend of the detection signals as the concentration increased), whereas the lower detection limit of bisulfite sequencing PCR is 0.5% methylation (linear relationships seldom occurred even though the sequencing depth was greater than ten thousand X). Take the detection results of two CpG sites in the sequence of SEQ ID NO.5 for example. (Information about the two sites is shown in FIG. 2.) The LOQ of the two CpG sites as detected by the method of the invention is shown in FIG. 3, with the average sequencing depth of each gradient interval being 100-200×. The LOQ of the same two CpG sites as detected by bisulfite sequencing PCR is shown in FIG. 4, with the average sequencing depth of each gradient interval being greater than 9500×.

(2) The detection method of the present invention has a lower-LOD (limit of detection) hypothesis than bisulfite sequencing PCR: Take the two CpG sites ch1_63795447 (−) and chr1_6379497 (−) in the sequence of SEQ ID NO.7 for example. At the LOD gradient of 2 copies, the detection method of the invention can detect the methylation states of the two sites consistently at a sequencing depth of several thousand X, but bisulfite sequencing PCR cannot, even at a sequencing depth of ˜40000× (the detected C was in the interval of sequencing noise), as detailed in TABLE 15:

TABLE 15
LOD hypotheses of the detection method of the present invention and of BSP
2 copies

| | BSP | | | | | |
|---|---|---|---|---|---|---|
| | | Sequencing | Sequencing | Detection method of the present invention | | |
| | | amount of site | amount of site | | C% of site | C% of site |
| | | 1, i.e., | 2, i.e., | | 1, i.e., | 2, i.e., |
| | Sequencing | Chr1_63795447 | Chr1_63795497 | Sequencing | Chr1_63795447 | Chr1_63795497 |
| | depth | (—) | (—) | depth | (—) | (—) |
| Replication | 38969 | 4 | 8 | 2002 | 100% | 99.92% |
| 1 | | | | | | |
| Replication | 47643 | 1 | 2 | 4131 | 99.95% | 100% |
| 2 | | | | | | |

### Embodiment 4

In this embodiment, the methylation levels of the eight sequences of SEQ ID NO.1-SEQ ID NO.8 in three clinical lung cancer tissue samples and three control tissue samples were detected separately by the detection method of the present invention and bisulfite sequencing PCR. The detection steps are as follows:

S1. Genomic DNA was extracted from the clinical lung cancer tissue samples and the control tissue samples, and the extracted DNA was processed with a bisulfite. After that, methylation-dependent multiplex PCR amplification was performed on the processed DNA.

More specifically, genomic DNA was extracted from the samples with the Qiagen-QIAamp-DNA-FFPE tissue kit (Qiagen, Cat #56404). The extraction steps were carried out according to the kit user guide. The extracted DNA was processed with a bisulfite and subjected to methylation-dependent multiplex PCR amplification as described in relation to embodiment 3.

Steps S2 and S3 were the same as described in relation to embodiment 3.

According to the detection results, the detection method of the present invention can detect, at a sequencing depth of several thousand X, the significant difference in methylation between the lung cancer samples and normal samples, but bisulfite sequencing PCR cannot. Take the detection results of two CpG sites (CpG site 1: ch5_40681550 and CpG site 2: ch5_40681569) in the sequence of SEQ ID NO.5 for example. (The two CpG sites are known to have relatively high methylation levels in lung cancer tissues.) The detection results of CpG site 1 ch5_40681550 are shown in FIG. 5, and those of CpG site 2 ch5_40681569 in FIG. 6. It can be seen in FIG. 5 and FIG. 6 that the method of the invention can find the significant difference of the detected sites in the lung cancer tissue samples at a sequencing depth of 2000×, and that BSP cannot effectively detect the difference between the normal samples and lung cancer samples even at a ten-times-greater sequencing depth, i.e., at 20000×.

### Comparative Example 1

The DNA methylation detection method in this comparative example is the same as the DNA methylation detection method in embodiment 3 except that the methylation-dependent multiplex PCR amplification in step S1 used a Q5 enzyme instead of the Phusion DNA polymerase, the Q5 enzyme being used in the same amount as the Phusion DNA polymerase. The methylation-dependent multiplex PCR amplification in the detection method of the present invention uses a high-fidelity enzyme selected from the group consisting of a Phusion DNA polymerase (e.g., the Phusion Hot Start II DNA Polymerase or the Phusion U Hot Start DNA Polymerase), a Q5 enzyme (e.g., the Q5® Hot Start High-Fidelity DNA Polymerase or the Q5U® Hot Start High-Fidelity DNA Polymerase), a Hieff enzyme (e.g., the Hieff NGS® HG Hot Start Multiplex PCR Enzyme), a KAPA DNA polymerase (e.g., the KAPA HiFi Uracil+ Kit or the KAPA2G Fast Hot Start DNA Polymerase), a Pfu enzyme, and a SuperFi enzyme. All the aforesaid high-fidelity enzymes can produce relatively good multiplex amplification results. A Phusion DNA polymerase, in particular, can produce the best multiplex amplification results. This comparative example was intended to compare the multiplex amplification results of a Q5 enzyme (preferably Q5® Hot Start High-Fidelity DNA Polymerase) with those of a Phusion DNA polymerase (preferably the Phusion U Hot Start DNA Polymerase).

Methylation-dependent multiplex PCR amplification was performed on 50 different genes in a 10%-methylation cfDNA mock standard by the methylation-dependent multiplex PCR method used in the DNA methylation detection method of this comparative example and by the methylation-dependent multiplex PCR method used in the DNA methylation detection method of embodiment 3, separately. The PCR detection conditions were the same: 98° C. for 30s; followed by 18 cycles each starting with 98° C. for 15 s and continuing with 60° ° C. for 2 min and then with 72° C. for 1 min; followed by 72° C. for 15 min.

An analysis was conducted with 2100 Bioanalyzer. Dimer percentages were calculated according to the ranges of fragment lengths. The dimer percentages of the amplification products of the two amplification methods were compared.

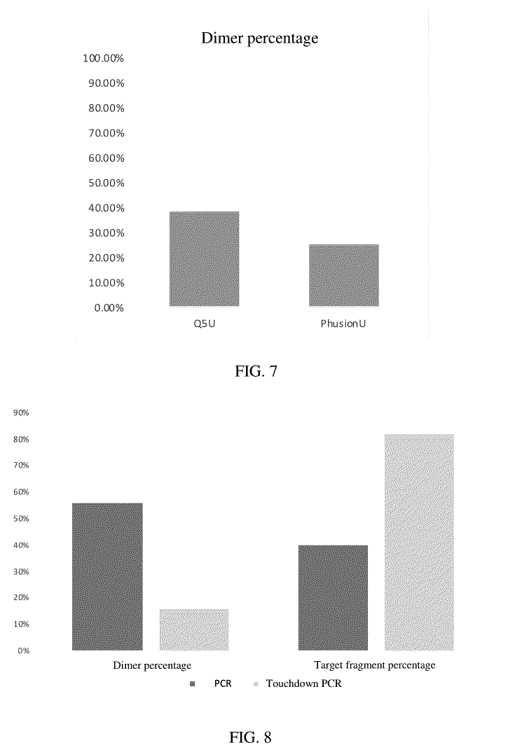

As shown by the detection results in FIG. 7, using a Phusion DNA polymerase in methylation-dependent multiplex PCR amplification (i.e., the methylation-dependent multiplex PCR method used in the detection method of embodiment 3) can reduce the dimer percentage of the amplification product more effectively than using a Q5 enzyme. A lower dimer percentage makes it easier to carry out subsequent detection on the multiplex PCR amplification product.

### Comparative Example 2

The DNA methylation detection method in this comparative example is the same as the DNA methylation detection method in embodiment 3 except that the multiplex PCR amplification in step S1 used a conventional PCR method whose reaction process is shown in TABLE 16:

TABLE 16
Reaction process of a conventional PCR method

| | | |
|---|---|---|
| Temperature | Time | Number of cycles |

| | | | |
|---|---|---|---|
| 98° C. | 30 | s | 1 |
| 98° C. | 15 | s | 30 |
| 60° C. | 15 | s | |
| 72° C. | 15 | s | |
| 72° C. | 5 | min | 1 |

| | | |
|---|---|---|
| 4° C. | For as long as needed | — |

Methylation-dependent multiplex PCR amplification was performed on 50 different genes in a 100%-methylation cfDNA mock standard by the methylation-dependent multiplex PCR method used in the DNA methylation detection method of this comparative example and by the methylation-dependent multiplex PCR method used in the DNA methylation detection method of embodiment 3, separately. The primer dimer percentages and target sequencing percentages of the amplification products of the two amplification methods were compared.

As shown by the detection results in FIG. 8 (in which “Normal PCR” refers to the conventional PCR method, and “Touchdown PCR” refers to the touchdown PCR method used in the present invention), performing methylation-dependent multiplex PCR amplification by a touchdown PCR method (i.e., the methylation-dependent multiplex PCR method used in the detection method of the invention) can reduce the dimer percentage of the amplification product more effectively (by up to 50 percentage points) than by a conventional PCR method. A lower dimer percentage makes it easier to carry out subsequent detection on the multiplex PCR amplification product and can increase the target gene sequencing percentage (the target gene sequencing percentage was increased by about 40 percentage points).

### Comparative Example 3

The DNA methylation detection method in this comparative example is the same as the DNA methylation detection method in embodiment 3 except that the T4 endonuclease-processed methylation-dependent multiplex PCR amplification product was purified in step S2 with Smart Beads instead of the XP Beads. The steps of purifying the multiplex PCR amplification product with Smart Beads are as follows: adding 50 μL of diethyl pyrocarbonate (DEPC) into 50 μL of PCR product→allowing the Smarter magnetic beads to reach equilibrium at room temperature for 30 min→mixing the beads thoroughly, and then mixing 220 μL (2.2×) of the beads with the sample by a vortex mixer→incubation at room temperature for 5 min→simple centrifugation, followed by placement on the magnetic base for magnetic attraction for 5 min, or until the liquid becomes clear, followed by removal of the supernatant by suction→rinsing with 200 μL of 80% ethanol for 30 s, and then removing the supernatant→rinsing with 200 μL of 80% ethanol for 30 s, and then removing the supernatant→simple centrifugation, followed by placement on the magnetic base until the liquid becomes clear, followed by removal of excessive liquid by suction→opening the lid, allowing the contents of the tube to dry until the magnetic bead surface is no longer reflective→adding 55 μL of EB for elution, followed by a thorough mix and then incubation at room temperature for 5 min→simple centrifugation, followed by placement on the magnetic base for magnetic attraction for 3-5 min, or until the liquid becomes clear, followed by collection of the supernatant (A total of 50 μL was collected. 2 μL was taken from each of a number of selected samples in order to carry out a Qubit test, and all such samples were subsequently replenished to 50 μL.).

The methylation-dependent multiplex PCR amplification products of 50 different genes in a 10%-methylation cfDNA mock standard were purified by the purification method used in the DNA methylation detection method of this comparative example and by the purification method used in the DNA methylation detection method of embodiment 3, separately. The dimer percentages of the amplification products corresponding respectively to the two purification methods were compared.

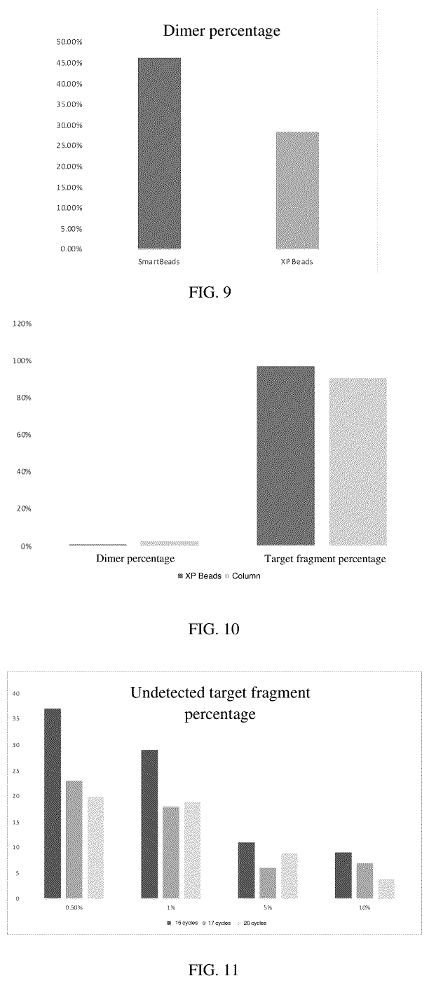

As shown by the detection results in FIG. 9, using XP Beads to purify a methylation-dependent multiplex PCR amplification product (the purification method used in the detection method of the present invention) can reduce the dimer percentage of the amplification product more effectively than using Smart Beads. A lower dimer percentage makes it easier to carry out subsequent detection on the purified multiplex PCR amplification product.

### Comparative Example 4

The DNA methylation detection method in this comparative example is the same as the DNA methylation detection method in embodiment 3 except that the T4 endonuclease-processed methylation-dependent multiplex PCR amplification product was purified in step S2 with a purification column instead of the XP Beads. The steps of purifying the multiplex PCR amplification product with a purification column (made by QIAGEN) are as follows: adding a binding solution into the PCR product, and mixing thoroughly by inverting the tube several times, wherein the volume of the binding solution is five times that of the PCR product→transferring into a DNA recovery column, allowing to rest at room temperature for 2 min, followed by centrifugation at 8,000 rpm for Imin→pouring out the waste liquid in the collection tube→repeating once→adding a wash solution to perform elution once, followed by centrifugation at 12,000 rpm for 1 min→adding 50 μL of elution buffer, allowing to rest for 2 min, followed by centrifugation at 12,000 rpm for 1 min.

The methylation-dependent multiplex PCR amplification products of 50 different genes in a 10%-methylation cfDNA mock standard were purified by the purification method used in the DNA methylation detection method of this comparative example and by the purification method used in the DNA methylation detection method of embodiment 3, separately. The dimer percentages and target sequencing percentages of the amplification products corresponding respectively to the two purification methods were compared.

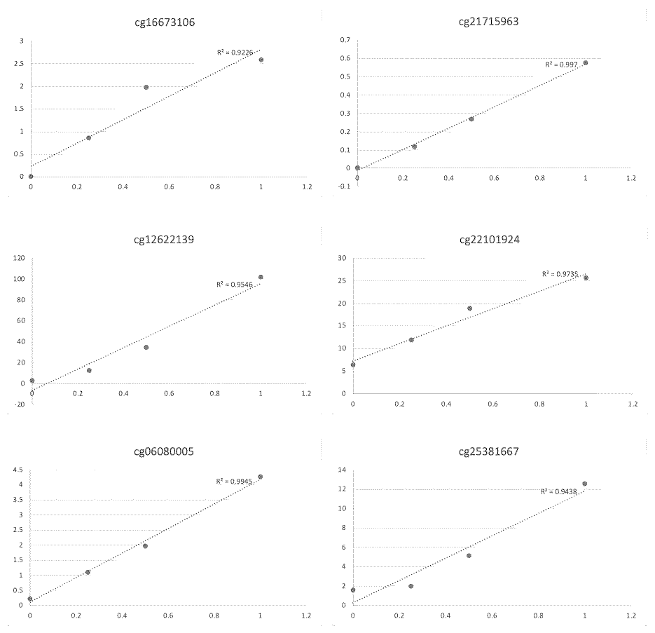

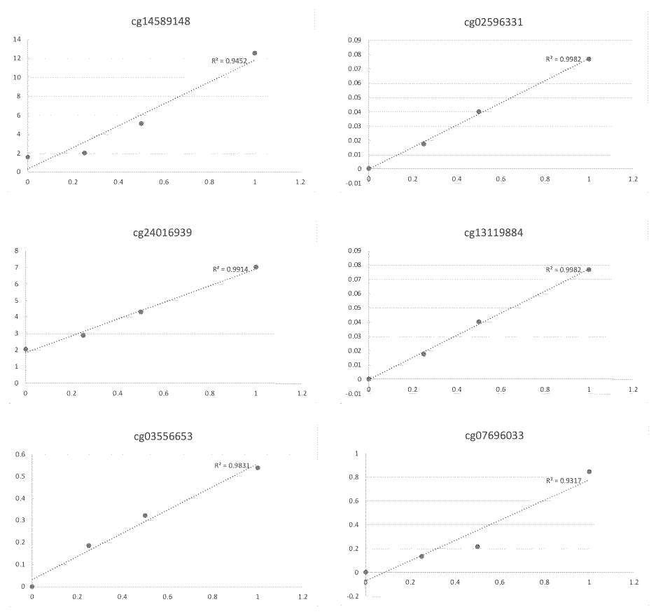

As shown by the detection results in FIG. 10 (in which “Column” refers to purification with a purification column), using XP Beads to purify a methylation-dependent multiplex PCR amplification product (the purification method used in the detection method of the present invention) can reduce the dimer percentage of the amplification product more effectively than using a purification column. A lower dimer percentage makes it easier to carry out subsequent detection on the purified multiplex PCR amplification product and can increase the target gene sequencing percentage.

### Comparative Example 5

The DNA methylation detection method in this comparative example is the same as the DNA methylation detection method in embodiment 3 except for the number of methylation-dependent multiplex PCR amplification cycles in step S1. Multiplex PCR amplification in this comparative example was performed under two different conditions: (1) 98° ° C. for 30 s; followed by 10 cycles each starting with 98° ° C. for 15 s and continuing with 65° C. (or a temperature successively lowered therefrom by 0.5° C. after each cycle) for 15 s and then with 72° C. for 15 s; followed by 17 cycles each starting with 98° C. for 15 s and continuing with 60° C. for 15 s and then with 72° C. for 15 s; followed by 72° C. for 15 min; and (2) 98° C. for 30 s; followed by 10 cycles each starting with 98° C. for 15 s and continuing with 65° C. (or a temperature successively lowered therefrom by 0.5° ° C. after each cycle) for 15 s and then with 72° C. for 15 s; followed by 20 cycles each starting with 98° C. for 15 s and continuing with 60° C. for 15 s and then with 72° C. for 15 s; followed by 72° C. for 15 min. The detection method of the present invention can provide detection when the number of methylation-dependent multiplex PCR amplification cycles ranges from 15 to 20. In particular, the most target genes can be successfully detected when the number of methylation-dependent multiplex PCR amplification cycles is 15.

Methylation-dependent multiplex PCR was performed on 153 different genes in differently methylated (i.e., 0.5%, 1%, 5%, and 10%-methylation) cfDNA mock standards by the methylation-dependent multiplex PCR methods used respectively in the two DNA methylation detection methods of this comparative example and the DNA methylation detection method of embodiment 3 of the present invention, followed by sequencing, an analysis, and a comparison. The detection results in FIG. 11 show that, under the conditions of 0.5%, 1%, 5%, and 10% methylation, the number of cycles being 15 leads to successful detection of significantly more target genes than the number of cycles being 17 or 20 and corresponds to the least undetected target genes.

### Embodiment 5

This embodiment was intended to evaluate the detection effect of the DNA methylation detection method of the present invention (i.e., the MeDAS method) on human-cell-line genomic DNA of different methylation levels. The steps of this embodiment are as follows:

(1) Preparation of genomic DNA standards of different methylation levels: The 0% and 100%-methylation human genomic DNA standards were purchased from Zymo (Cat #D5014). The 0%-methylation standard was sourced from the HCT116 [DNMT1 (−/−) DNMT3b (−/−)] cell line, and the 100%-methylation standard was sourced from a methyltransferase-processed product of the 0%-methylation standard and was verified by sequencing. The 25% and 50%-methylation standards were produced by mixing the 0% and 100%-methylation standards in appropriate proportions.

(2) In this embodiment, methylation-dependent multiplex PCR primers were designed for 32 randomly selected CpG sites in the genome, as shown in the following table:

| | | | SEQ |
|---|---|---|---|
| | | | ID |
| CpG site | Primer design | | NO. |
| cg16673106 | MF1 | GAGTAGGTAGAGTCGGGGAC | 49 |
| | MR1 | ACTTCTACAACTAAATTCGAAAACGTC | 50 |
| cg14477452 | MF1 | TTTTGGGCGTAGAGTAGCGGTT | 51 |
| | MR1 | CTCTAAATCTCGATAAAACTCGCAT | 52 |
| cg12622139 | MF1 | ACGCGCGTCGGAGGATTTC | 53 |
| | MR1 | AATCGCCACCCGAACGAACG | 54 |
| cg25497529 | MF1 | TTGTAGGTTAGGGAGATTACGTTT | 55 |
| | MR1 | CTATCTCTATATCTCTATCTCCCG | 56 |
| cg06080005 | MF1 | GCGGTTTAGTATCGGTGGGAGATCGT | 57 |
| | MR1 | TAAACCAAAATCGAAAATCGCGACC | 58 |
| cg14242042 | MF1 | TTACGTCGGAGGAGGTATTAACGAGA | 59 |
| | MR1 | CTTAAAAATCTACCCACAACAACATCGAAACG | 60 |
| cg21715963 | MF1 | TTTTTTTAGATACGTGCGGT | 61 |
| | MR1 | CCTCTCTACTAACTAAACCCCTTTATACTA | 62 |
| cg07959338 | MF1 | TCGGTTAATTAATTTGGGAGGCGAAA | 63 |
| | MR1 | GACGCGATACGACTCACTCCGCTA | 64 |
| cg22101924 | MF1 | GTAGTTTCGGTAGAGGCGTTT | 65 |
| | MR1 | CTACTAAATTACTAAACGAATCGAAA | 66 |
| cg21542248 | MF1 | GGTTTAAAATTCGAGAAAATAACG | 67 |
| | MR1 | ACGCCTCAAAAACTCCGAAA | 68 |
| cg25381667 | MF1 | GAGTTATAGTGAGTCGGTTACGTAAATAGCGA | 69 |
| | MR1 | ATTACTTATCAACGCCGACAAACTACCGCT | 70 |
| cg25999722 | MF1 | TTACGTTATTGGTTGGAGGGTGCG | 71 |
| | MR1 | CTAACTAAACCGCGCGAACG | 72 |
| cg14589148 | MF1 | AGAGTTATAGTGAGTCGGTTACGTAAATAGCG | 73 |
| | MR1 | CTTATTACTTATCAACGCCGACAAACTACCGC | 74 |
| cg24496978 | MF1 | GTAGTTGAGTTGTAGGATGTAAGCG | 75 |
| | MR1 | CCAAACCCTACTAAAACCCG | 76 |
| cg24016939 | MF1 | TTTAAGGTAGGGGATTTTCGG | 77 |
| | MR1 | AAAATCCTATCAACCCTTTAAAACTCGAA | 78 |
| cg11679177 | MF1 | CGTAGGTAGGTGAAAGTAGGC | 79 |
| | MR1 | GAACTTCCTCTCTATTCCCCC | 80 |
| cg03556653 | MF1 | TTATAGAAATTAGGAGGCGCGTA | 81 |
| | MR1 | GCAACTCCTCTAAACCGAAC | 82 |
| cg02596331 | MF1 | AGATATTTTTAAAAGGTAGCGAAA | 83 |
| | MR1 | ACACTACCTCTCCAACATAAAAACG | 84 |
| cg13119884 | MF1 | AGTTATAACGTATTGAAGTTGCG | 85 |
| | MR1 | TTATTATAAACGCTCAAATRGAAAAA | 86 |
| cg23516634 | MF1 | GTAGGGGTAGGGATTACGGT | 87 |
| | MR1 | TTAATCTCCTAAACAACAATATATAACCGA | 88 |
| cg07696033 | MF1 | GTTTTGAGTCGTACGCGTTG | 89 |
| | MR1 | CTTAAAAACCAAAATCCTCCGA | 90 |
| cg13552710 | MF1 | TAGTAGCGCGGAGTTGGTTT | 91 |
| | MR1 | ACTACTACCACCGCTACCGC | 92 |
| cg24876786 | MF1 | TAAAGGGAATGTGGGGTTTC | 93 |
| | MR1 | CGCTTCCTTCTCTTAAACTAAAAA | 94 |
| cg15811719 | MF1 | AGAATTTCGTTTAGGGTAGGGC | 95 |
| | MR1 | GATACGAATTTCTAATACGCTAACC | 96 |
| cg06123396 | MF1 | ATTTAGATTGTTAGTAGCGGG | 97 |
| | MR1 | AAAACACAAACCGAAACAAAAC | 98 |
| cg00901765 | MF1 | TATAATAAGGTGATGTGTTAGGAAGC | 99 |
| | MR1 | AAAACAAACAAAAACAAACGTA | 100 |
| cg12180984 | MF1 | AAAGAGGGGAGAGAGTTCGC | 101 |
| | MR1 | CTATATCCTCGACCCCGATT | 102 |
| cg23753247 | MF1 | AGTAATCGTAGGGTAGTGGACGGGG | 103 |
| | MR1 | CTCTAAAACGAACTACCCTTAAACGCGCC | 104 |
| cg07944863 | MF1 | AGGTACGTGATGAAATTTTGGT | 105 |
| | MR1 | GAAATTTAACGCCCAATATAAACC | 106 |
| cg04234680 | MF1 | TCGTGGAAGGAAGTACGTTT | 107 |
| | MR1 | CGACATAAAATTCAACTAAATAACCG | 108 |
| cg16712637 | MF1 | GAATTAGAGCGATTCGGGAC | 109 |
| | MR1 | GTCAAAACTCCCGCTTCATATT | 110 |
| cg14603466 | MF1 | TAGTGAGTAGAGAAAGACGTACGAAA | 111 |
| | MR1 | GCCTATCTACACCCTATCGCC | 112 |

(3) The 0%, 25%, 50%, and 100%-methylation standards were processed with a bisulfite and underwent methylation-dependent multiplex PCR amplification according to the methods used in embodiment 3. Steps S2 and S3 were the same as described in relation to embodiment 3.

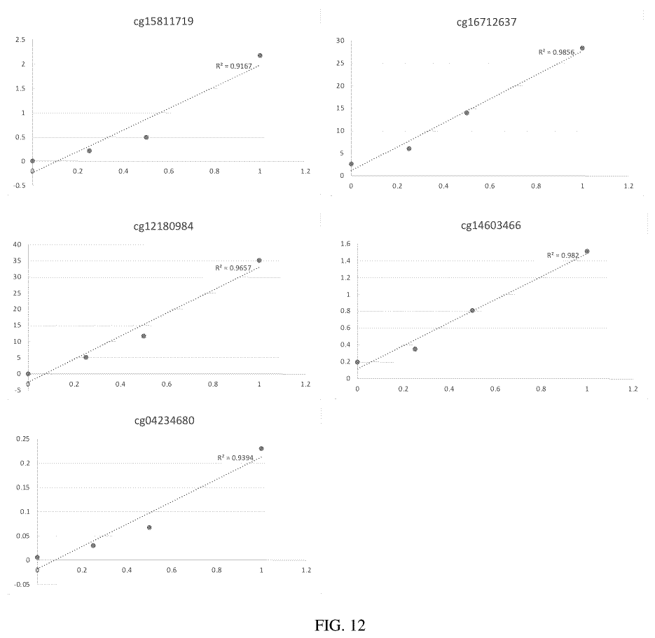

As shown by the detection results in FIG. 12, the detection method of the present invention enables satisfactory linear regression (R²>0.8) of signals detected from those standards that, in addition to having different methylation concentrations respectively, included the following 17 target sites: cg16673106, cg25381667, cg24016939, cg22101924, cg21715963, cg16712637, cg15811719, cg14603466, cg14589148, cg13119884, cg12622139, cg12180984, cg07696033, cg06080005, cg04234680, cg03556653, and cg02596331. The detection results show that the detection method of the invention can be used to carry out high-throughput effective detection of the methylation levels of certain target sits in cell-line genomic DNA.

### Embodiment 6

This embodiment was intended to evaluate the detection effect of the DNA methylation detection method of the present invention (i.e., the MeDAS method) on markers in gastric cancer patients' blood samples. The steps of this embodiment are as follows:
1. Whole-blood processing: In this embodiment, the plasma samples of 152 normal people (in whom gastric cancer was not found by gastroscopy) and of 109 gastric cancer patients were detected.
1.1 10 mL of whole blood was collected from each subject with an anticoagulant-treated vacuum EDTA K2 blood collection tube (BD, Cat #367525) and was sufficiently mixed to prevent hemolysis. The whole blood samples underwent plasma separation within 4-6 hours after they were taken.
1.2 The whole-blood samples were centrifuged at low speed (1600 g) and 4° C. for 15 min. The plasma in the top layer was carefully collected by suction, without the buffy coat being collected. The plasma obtained was centrifuged at high speed (16000 g) and 4° C. for 10 min to produce the desired sample plasma.
2. cfDNA extraction from the plasma

Extraction method: The plasma DNA was extracted according to the steps specified in the user guide of the MagMAX™ Cell-Free DNA Isolation Kit of Thermo Fisher.
3. Bisulfite-Mediated Conversion of the Extracted cfDNA

The extracted DNA was subjected to bisulfite-mediated conversion to deaminate the unmethylated cytosine in the DNA and thereby conversion the unmethylated cytosine into uracil while the methylated cytosine was kept unchanged. The product obtained was bisulfite-converted DNA. The conversion was carried out according to the protocol of Zymo DNA Methylation-Direct MagPrep, with the amount of input cfDNA being 5-20 ng, or preferably 10 ng as in this embodiment. Multiplex methylation amplification was then performed on all the bisulfite-converted product.
4. Performing multiplex methylation amplification on specific markers in the converted cfDNA

Multiplex methylation amplification was performed on all the conversion product. The reaction constituents included: a combination of primers for 103 gastric cancer differentiation markers obtained by tissue screening, the final concentration of each primer being 50-200 nM; magnesium ions at a concentration of 2-5 mM, or preferably at 3 mM as in this embodiment; and a dNTP mix at a concentration of 100-600 μM, or preferably at 200 μM as in this embodiment. The enzyme used in the reaction was KAPA2G Fast Multiplex PCR Kit (Roche, Cat #KK5802). The reaction conditions were: 95° ° C. for 5 min for initial denaturation, followed by 15-30 or preferably 20 cycles each starting with 95° C. for 15 s for denaturation and continuing with 58-66° ° C., or preferably 63° ° C. as in this embodiment, for 4 min for annealing.

The multiplex reaction system was designed as follows:

| Constituent | Volume (μL) | Final concentration |
|---|---|---|
| 2 × KAPA2G Fast Multiple Mix | 25 | 1X |
| Primer mix (500 nM each) | 5 | 50 nM each |
| Template | 20 | |

5. Library construction for the multiplex methylation amplification product

Library construction for the multiplex methylation amplification product was performed with NEB NEBNext® Ultra™ II DNA Library Prep Kit for Illumina® and NEBNext® Multiplex Oligos for Illumina® (Dual Index Primers Set 1). Please refer to the kit user guides for the operational details.
6. The samples were pooled in accordance with the principle of equal amounts and then applied to the sequencing machine in order to be sequenced and then statistically analyzed.

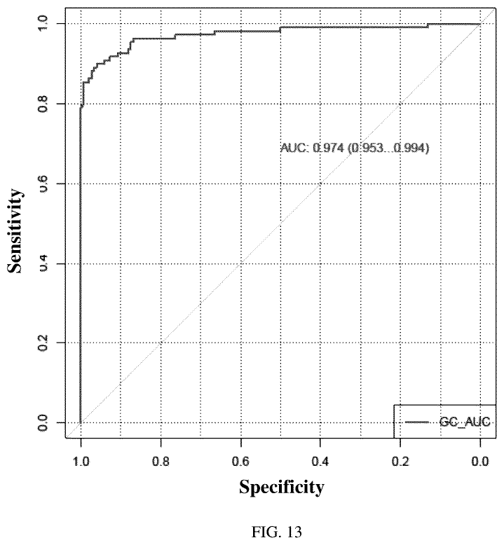

Experimental results: All the 103 markers were used in model construction according to the random forest model and were subsequently analyzed. Cleavage in a ratio of 7:3 was repeated 100 times. Given 99% specificity, the test set showed phase-1 detection sensitivity of 87.5%, phase-2 detection sensitivity of 92.9%, phase-3 detection sensitivity of 77.8%, and phase-4 detection sensitivity of 86.7%. The overall detection sensitivity toward all the colorectal cancer samples was 85.3%. The overall AUC (area under the receiver operating characteristic (ROC) curve) was 0.974. The experimental results indicate that the markers can be used together with the method of the present invention in early screening of gastric cancer. The ROC curve is shown in FIG. 13.

### Embodiment 7

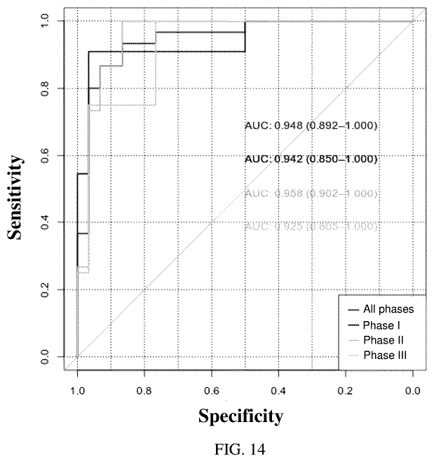

This embodiment was intended to evaluate the detection effect of the DNA methylation detection method of the present invention (i.e., the MeDAS method) on markers in breast cancer patients' blood samples, in order to determine whether the markers can be used in conjunction with the detection method in early screening and early diagnosis of breast cancer. For more details about the steps of this embodiment, please refer to embodiment 2 of the invention. The plasma samples of 30 normal people and of 30 breast cancer patients were detected. All the 54 methylation markers obtained by tissue screening were used in model construction according to the random forest model and were subsequently analyzed. Cleavage in a ratio of 7:3 was repeated 100 times. Given 99% specificity, the 11 phase-1 samples in the test set showed 54.5% detection sensitivity, the 15 phase-2 samples showed 26.7% detection sensitivity, and the four phase-3 samples showed 25% detection sensitivity. The overall detection sensitivity toward all the breast cancer samples was 36.7%. The overall AUC was 0.948. The experimental results indicate that the markers can be used together with the method of the invention in early screening of breast cancer. The ROC curve is shown in FIG. 14.

The technical features of the foregoing embodiments can be arbitrarily combined. For the sake of brevity, however, this specification does not describe all the possible combinations of the technical features of the embodiments. All such combinations shall be construed as falling within the scope of the specification, provided that the technical features 5 combined do not conflict with one another.

The embodiments described above disclose only a few modes of implementing the present invention. The relatively specific and detailed description of those embodiments shall not be understood as restrictive of the scope of patent protection for the invention. It should be pointed out that a person of ordinary skill in the art may make various changes and improvements without departing from the concept of the invention, and that all such changes and improvements shall fall within the scope of patent protection for the invention. The scope of patent protection for the invention is defined by the appended claims.

1. A methylation detection method for genomic DNA or cell-free DNA molecules in a biological sample, characterized by:
obtaining a converted DNA molecule through a chemical and/or enzymatic reaction; performing a methylation-specific, i.e., methylation-dependent, multiplex polymerase chain reaction (PCR), through multiple pairs of methylation-specific primers, on one or more methylated areas of one or more genes in the converted DNA molecule serving as a substrate, in order to obtain a PCR amplification product; and
detecting methylation levels of different fragments of the amplification product.

2. The methylation detection method of claim 1, comprising the steps of:
S1. extracting genomic DNA or cell-free DNA from the biological sample, processing the extracted DNA with the chemical and/or enzymatic reaction to obtain the converted DNA molecule, and performing the methylation-specific, i.e., methylation-dependent, multiplex PCR on the converted DNA molecule through the methylation-specific primers, wherein different said methylation-specific PCR primers are used on different said genes or different methylated sites;
S2. processing and purification of the multiplex PCR product: processing the multiplex PCR product obtained from the step S1 with an endonuclease or DNA-fragment-sorting magnetic beads in order to obtain a processed product, and purifying the processed product in order to obtain a purified product; and
S3. detecting the methylation levels of the purified product obtained from the step S2.

3. The methylation detection method of claim 2, wherein the biological sample comprises a tissue sample, a cell sample, or a fluid sample of an organism and is preferably selected from the group consisting of blood, blood serum, blood plasma, a vitreous body, sputum, urine, tear, sweat, and saliva.

4. The methylation detection method of claim 2, wherein the genomic DNA or the cell-free DNA extracted from the biological sample is subjected to a chemical and/or enzymatic conversion.

5. The methylation detection method of claim 1 or 2, wherein the chemical and/or enzymatic reaction used for DNA conversion comprises a chemical method (e.g., a bisulfite method), an enzymatic method (e.g., through a ten-eleven translocation (TET) enzyme or an apolipoprotein B mRNA editing enzyme, catalytic polypeptide (APOBEC) enzyme), or a mixed chemical-enzymatic method (e.g., through a TET enzyme and pyridine borane).

6. The methylation detection method of claim 5, wherein the chemical and/or enzymatic method converts unmethylated cytosines in a DNA molecule into uracils and preserves 5′-methylcytosines/5′-hydroxylcytosines in the DNA molecule in order to obtain the converted DNA molecule.

7. The methylation detection method of claim 5, wherein the mixed chemical-enzymatic method converts 5′-methylcytosines/5′-hydroxylcytosines in a DNA molecule into dihydrouracil and preserves unmethylated cytosines in the DNA molecule in order to obtain the converted DNA molecule.

8. The methylation detection method of claim 1 or 2, further comprising processing and purifying the multiplex PCR product by: processing the obtained multiplex PCR product with an endonuclease or DNA-fragment-sorting magnetic beads in order to obtain a processed product, and purifying the processed product.

9. The methylation detection method of claim 2, wherein reaction conditions of the methylation-dependent multiplex PCR in the step S1 are: 98° ° C. for 30 s to 5 min; followed by 10-25 cycles each starting with 98° C. for 15 s and continuing with 60±10° C. for 15 s to 10 min and then with 68-72° C. for 15 s to 5 min; followed by 68-72° C. for 0 min to 15 min.

10. The methylation detection method of claim 9, wherein the reaction conditions of the methylation-dependent multiplex PCR in the step S1 are: 98° C. for 30 s; followed by 5-10 cycles each starting with 98° C. for 15 s and continuing with 65±3° C., or a temperature successively lowered therefrom by 0.2° C.-0.8° C. after each said cycle, for 15 s and then with 72° C. for 15 s; followed by 10-25 cycles each starting with 98° C. for 15 s and continuing with 60±10° C. for 15 s to 10 min and then with 68° C.-72° C. for 15 s to 5 min; followed by 72° C. for 0 min to 15 min.

11. The methylation detection method of claim 2, wherein the methylation-dependent multiplex PCR in the step S1 uses an enzyme selected from the group consisting of a Phusion DNA polymerase, a Q5 enzyme, a Hieff enzyme, a KAPA DNA polymerase, a Pfu enzyme, a SuperFi enzyme, and a corresponding uracil-compatible polymerase, preferably from the group consisting of a Phusion U enzyme, a KAPA U enzyme, a Q5U enzyme, and a HieffU enzyme.

12. The methylation detection method of claim 11, wherein the enzyme is the Phusion DNA polymerase.

13. The methylation detection method of any of claims 1 to 11, wherein the methylation-dependent multiplex PCR in the step S1 is performed on one or more different said genes and/or one or more different reference sequences simultaneously.

14. The methylation detection method of claim 13, wherein the one or more different reference sequences are at least one selected from the group consisting of:
reference sequence 1: a sequence in the EPHA3 gene that corresponds to a fragment obtained by PCR amplification with SEQ ID NO.25 and SEQ ID NO.26 serving as specific PCR primers;
reference sequence 2: a sequence in the KBTBD4 gene that corresponds to a fragment obtained by PCR amplification with SEQ ID NO.27 and SEQ ID NO.28 serving as specific PCR primers;
reference sequence 3: a sequence in the PLEKHF1 gene that corresponds to a fragment obtained by PCR amplification with SEQ ID NO.29 and SEQ ID NO.30 serving as specific PCR primers; and
reference sequence 4: a sequence in the SYT10 gene that corresponds to a fragment obtained by PCR amplification with SEQ ID NO.31 and SEQ ID NO.32 serving as specific PCR primers.

15. The methylation detection method of claim 14 wherein the one or more different reference sequences are at least two selected from the group consisting of the reference sequences 1-4.

16. The methylation detection method of claim 8, wherein the one or more different genes have at least one sequence selected from the group consisting of SEQ ID NO.1-SEQ ID NO.8.

17. The methylation detection method of claim 13, wherein the methylation-dependent multiplex PCR is performed on at least one sequence selected from the group consisting of SEQ ID NO.1-SEQ ID NO.8 under the reaction conditions of: 98° C. for 30 s; followed by 10 cycles each starting with 98° C. for 15 s and continuing with 65±3° C., or a temperature successively lowered therefrom by 0.2° C.-0.8° ° C. after each said cycle, for 15 s and then with 72° ° C. for 15 s; followed by 15-20 cycles each starting with 98° C. for 15 s and continuing with 60±3° C. for 15 s and then with 72° C. for 15 s; followed by 72° C. for 15 min.

18. The methylation detection method of claim 2, wherein the processing with the endonuclease in the step S2 is carried out at 37±1° ° C. for 10 min to 15 min, and the endonuclease has a final concentration of 1-10 U/μL in a processing system of the step S2.

19. The methylation detection method of claim 18, wherein the endonuclease is a T4 endonuclease.

20. The methylation detection method of claim 2, wherein the DNA-fragment-sorting magnetic beads in the step S2 are XP magnetic beads.

21. The methylation detection method of claim 2, wherein the purification in the step S2 is purification with magnetic beads.

22. The methylation detection method of claim 21, wherein the magnetic beads are XP magnetic beads.

23. The methylation detection method of claim 2, wherein the methylation levels of the purified product obtained from the step S2 are detected in the step S3 by a sequencing method or a universal array method.

24. The methylation detection method of claim 23, wherein the sequencing method comprises second-generation sequencing and third-generation sequencing.

25. The methylation detection method of claim 23, wherein the sequencing method in the step S3 comprises the steps of: (1) repairing, and simultaneously adding a base A to, a 3′ end of the purified product obtained from the step S2; (2) ligating a sequencing adapter to a product obtained from the step (1); (3) performing PCR amplification, through an adapter primer, on a ligation product obtained from the step (2) in order to obtain a sequencing library; and (4) pooling, according to a same mole number, said libraries prepared from different said samples, and sequencing the pooled libraries.

26. A methylation detection kit for genomic DNA or circulating free DNA molecules in a biological sample, characterized in that the kit comprises polymerase chain reaction (PCR) primers specific to at least one sequence selected from the group consisting of SEQ ID NO.1-SEQ ID NO.8, wherein the primers are:
a forward primer of SEQ ID NO.9 and a reverse primer of SEQ ID NO.10, both serving as PCR primers specific to SEQ ID NO.1; and/or
a forward primer of SEQ ID NO.11 and a reverse primer of SEQ ID NO.12, both serving as PCR primers specific to SEQ ID NO.2; and/or
a forward primer of SEQ ID NO.13 and a reverse primer of SEQ ID NO.14, both serving as PCR primers specific to SEQ ID NO.3; and/or
a forward primer of SEQ ID NO.15 and a reverse primer of SEQ ID NO.16, both serving as PCR primers specific to SEQ ID NO.4; and/or
a forward primer of SEQ ID NO.17 and a reverse primer of SEQ ID NO.18, both serving as PCR primers specific to SEQ ID NO.5; and/or
a forward primer of SEQ ID NO.19 and a reverse primer of SEQ ID NO.20, both serving as PCR primers specific to SEQ ID NO.6; and/or
a forward primer of SEQ ID NO.21 and a reverse primer of SEQ ID NO.22, both serving as PCR primers specific to SEQ ID NO.7; and/or
a forward primer of SEQ ID NO.23 and a reverse primer of SEQ ID NO.24, both serving as PCR primers specific to SEQ ID NO.8.

27. The methylation detection kit of claim 26, wherein the kit further comprises:
specific PCR primers composed of a forward primer of SEQ ID NO.25 and a reverse primer of SEQ ID NO.26; and/or
specific PCR primers composed of a forward primer of SEQ ID NO.27 and a reverse primer of SEQ ID NO.28; and/or
specific PCR primers composed of a forward primer of SEQ ID NO.29 and a reverse primer of SEQ ID NO.30; and/or
specific PCR primers composed of a forward primer of SEQ ID NO.31 and a reverse primer of SEQ ID NO.32.

28. The methylation detection kit of claim 26 or 27, wherein the kit further comprises a Phusion DNA polymerase, a T4 endonuclease, and/or DNA-purifying magnetic beads.

29. A methylation detection kit for genomic DNA or circulating free DNA molecules in a biological sample, characterized by comprising at least one pair of CpG site-specific primers selected from the group consisting of:
SEQ ID NO.49 and 50, both specific to cg16673106;
SEQ ID NO.51 and 52, both specific to cg14477452;
SEQ ID NO.53 and 54, both specific to cg12622139;
SEQ ID NO.55 and 56, both specific to cg25497529;
SEQ ID NO.57 and 58, both specific to cg06080005;
SEQ ID NO.59 and 60, both specific to cg14242042;
SEQ ID NO.61 and 62, both specific to cg21715963;
SEQ ID NO.64 and 65, both specific to cg07959338;
SEQ ID NO.66 and 67, both specific to cg22101924;
SEQ ID NO.68 and 69, both specific to cg21542248;
SEQ ID NO.70 and 71, both specific to cg25381667;
SEQ ID NO.72 and 73, both specific to cg25999722;
SEQ ID NO.74 and 75, both specific to cg14589148;
SEQ ID NO.76 and 77, both specific to cg24496978;
SEQ ID NO.78 and 79, both specific to cg24016939;
SEQ ID NO.80 and 81, both specific to cg11679177;
SEQ ID NO.82 and 83, both specific to cg03556653;
SEQ ID NO.84 and 85, both specific to cg02596331;
SEQ ID NO.86 and 87, both specific to cg13119884;
SEQ ID NO.88 and 89, both specific to cg23516634;
SEQ ID NO.90 and 91, both specific to cg07696033;
SEQ ID NO.92 and 93, both specific to cg13552710;
SEQ ID NO.94 and 95, both specific to cg24876786;
SEQ ID NO.96 and 97, both specific to cg15811719;
SEQ ID NO.98 and 99, both specific to cg06123396;
SEQ ID NO.100 and 101, both specific to cg00901765;
SEQ ID NO.102 and 103, both specific to cg12180984;
SEQ ID NO.104 and 105, both specific to co23753247;
SEQ ID NO.106 and 107, both specific to cg07944863;
SEQ ID NO.108 and 109, both specific to cg04234680;
SEQ ID NO.110 and 111, both specific to cg16712637; and
SEQ ID NO.112 and 113, both specific to cg14603466.
